(19) United States
(12) Patent Application Publication
(10) Pub. No.: US 2026/0357303 A1
(43) Pub. Date: Nov. 12, 2026

(54) IN SITU NADP BIOSYNTHESIS PROCESS FOR BIOBASED CHEMICAL PRODUCTION

(71) Applicant: INVIZYNE TECHNOLOGIES, INC., Monrovia, CA (US)

(72) Inventors: Tyler P. KORMAN, Sierra Madre, CA (US); Hong YU, Torrance, CA (US)

(73) Assignee: INVIZYNE TECHNOLOGIES, INC., Monrovia, CA (US)

(21) Appl. No.: 19/487,208

(22) PCT Filed: May 24, 2024
(86) PCT No.: PCT/US2024/031044

§ 371 Date: Nov. 24, 2025

Related U.S. Application Data
(60) Provisional application No. 63/468,911, filed on May 25, 2023.

(51) Int. Cl. C12P 7/16; C12N 9/12

(52) U.S. Cl. CPC C12P 7/16; C12N 9/1205; C12N 9/1241; C12Y 207/01023; C12Y 207/07001

(57) ABSTRACT

The present disclosure relates to multi-enzyme pathways for the in situ generation of the cofactor NADP from lower-cost reagents, such as nicotinate, NMN (nicotinamide mononucleotide), and NAD (nicotinamide adenine dinucleotide), and the use of these pathways in NADP-dependent cell-free systems for the biobased production of chemicals, such as isobutanol.

### CROSS REFERENCE TO RELATED APPLICATIONS

This application claims priority benefit to U.S. Provisional Application No. 63/486,911, filed May 25, 2023, which is hereby incorporated by reference herein.

### STATEMENT REGARDING FEDERALLY SPONSORED RESEARCH

The inventions disclosed herein were made with government support under grant award number DE-EE0008925 from the Bioenergy Technologies Office of the U.S. Department of Energy. The government has certain rights in the inventions.

### FIELD

The present disclosure relates to a multi-enzyme pathway for the in situ generation of the cofactor NADP from lower-cost reagents, such as nicotinate, NMN (nicotinamide mononucleotide), and NAD (nicotinamide adenine dinucleotide), and the use of this pathway in NADP-dependent cell-free systems for the biobased production of compounds, such as isobutanol.

### REFERENCE TO SEQUENCE LISTING

The official copy of the Sequence Listing is submitted concurrently with the specification via USPTO Patent Center as a WIPO Standard ST.26 formatted XML file with file name “15041-004WO1_ST26.xml”, a creation date of May 24, 2024, and a size of 60,700 bytes. This Sequence Listing filed via USPTO Patent Center is part of the specification and is incorporated in its entirety by reference herein.

### BACKGROUND

Nicotinamide adenine dinucleotide phosphate (NADP) functions as a coenzyme that is essential for the biosynthetic production of isobutanol. U.S. Pat. No. 10,760,103B2, which is hereby incorporated by reference herein for all purposes, describes a cell-free biosynthetic system that produces isobutanol from glucose in high-titer and high-yield. FIG. 1 depicts a schematic of a biosynthetic pathway for isobutanol production that includes a “molecular rheostat” as described in U.S. Pat. No. 10,760,103B2. During a 5-day biosynthetic process with continuous product extraction, the final titer of isobutanol produced by this cell-free system can reach about 275 g/L with a maximum productivity of 4 g/L/h at 95% yield.

NADP is an essential coenzyme for the isobutanol biosynthetic process of FIG. 1. NADP acts as a redox carrier that transfers electrons generated by glycolysis to isobutyraldehyde for the production of isobutanol and maintains the redox balance of the entire system. NADP is biosynthesized in nature typically from nicotinate (or nicotinamide), ribose (or xylose), ammonia and ATP (adenosine 5′-triphosphate). (See also e.g., Sherkhanov, S. et al. “Isobutanol production freed from biological limits using synthetic biochemistry,” Nat Commun. 11, 4292 (2020); Yang Y, et al., “NAD (+) metabolism: Bioenergetics, signaling and manipulation for therapy,” Biochim Biophys Acta. 2016 December; 1864 (12): 1787-1800).

The relatively high market price of NADP greatly affects the economics of this biosynthetic process for the production of isobutanol, and biosynthesis processes for other biobased chemical compounds, such as 3-methyl-1-butanol, leucine, and valine. It would be greatly advantageous to find lower cost alternatives for biosynthesis processes that utilize market-priced coenzyme reagent, NADP in the production biobased chemical compounds, such as isobutanol, 3-methyl-1-butanol, leucine, and valine.

### SUMMARY

The present disclosure relates generally to biosynthetic process that utilize alternative lower-cost, multi-enzymatic pathways for the in situ production of the coenzyme NADP from a variety of lower cost sources such as nicotinate, NMN (nicotinamide mononucleotide) or NAD (nicotinamide adenine dinucleotide) in a cell-free system. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides a cell-free process for NADP-dependent biosynthesis of a compound, the process comprising contacting in a cell-free solution under suitable reaction conditions: an NADP-dependent enzyme; a substrate for the NADP-dependent enzyme; ATP; and
(a) NAD, and a NAD kinase;
(b) NMN, a NMN adenyltransferase, and a NAD kinase; and/or
(c) ribose, nicotinate, NH⁴⁺, and a NAD kinase;
whereby, NADP is produced in the solution and used by the NADP-dependent enzyme in the conversion of the substrate to the compound or to a precursor of the compound.

In at least one embodiment, the cell-free solution does not contain any NADP or NADPH prior to contacting with (a) NAD, and a NAD kinase; (b) NMN, a NMN adenyltransferase, and a NAD kinase; and/or (c) ribose, nicotinate, NH⁴⁺, and a NAD kinase.

In at least one embodiment of the process, the cell-free solution does not comprise NADP or NADPH prior to contacting with (a) NAD, and a NAD kinase; (b) NMN, a NMN adenyltransferase, and a NAD kinase; and/or (c) ribose, nicotinate, NH⁴⁺, and a NAD kinase.

In at least one embodiment of the process, the NAD kinase is selected from an exemplary NAD kinase of Table 2, such as EcNadK (WP_001059169.1), GsppnK1 (WP_011232266.1), GsppnK2 (WP_011230331.1), BliNadK (WP_009329373.1), TkNadK (WP_011251074.1), TmNadK (WP_004082250.1), PhNadK (WP_010885160.1), AfNadK (WP_010879860.1), CsNadK (WP_009610251.1), AtNadK (WP_003518725.1), ToNadK (WP_012572594.1), UTR1 (NP_012583.1), MtNadK (WP_003408383.1), BsNadK (WP_106610845.1), or a mixture thereof.

In at least one embodiment of the process, the NMN adenyltransferase is selected from: an NMN adenyltransferase of Table 3, such as ScNma1 (NP_013432.1), ScNma2 (NP_011524.1), POF1 (NP_009883.1), RnNmnat2 (NP_001041507.1), BtNmnat1 (NP_001069302.1), BtNmnat2 (XP_005217345.1), MmNmnat1 (NP_001343286.1), MmNmnat2 (NP_780669.1), MmNmnat3 (NP_653116.1), HsNmnat1 (NP_001284707.1), HsNmnat2 (NP_055854.1), HsNmnat3 (NP_001307440.1), AtNmnat (NP_200392.3), or a mixture thereof.

In at least one embodiment of the process, the cell-free solution further comprises the enzymes: RbsK, Prs, PncB, NadD, and NadE; optionally, wherein the enzymes are GsRbsK (ALA70174.1), TkPrs (WP_011251185.1), EcPncB (WP_001307697.1), GsNadD (WP_053532206.1), and EcNadE (EFI2440128.1).

In at least one embodiment of the process, the cell-free solution further comprises:
(a) a precursor substrate, and one or more enzymes capable of converting the precursor substrate to the substrate for the NADP-dependent enzyme; and/or
(b) one or more enzymes capable of converting the precursor of the compound produced by the NADP-dependent enzyme to the compound.

In at least one embodiment of the process, the cell-free solution further comprises a precursor substrate selected from a C6 sugar, a C5 sugar, and a mixture thereof.

In at least one embodiment of the process, the compound produced is selected from isobutanol, 3-methyl-1-butanol, leucine, and valine.

In another embodiment, the present disclosure also provides a composition for NADP-dependent biosynthesis comprising a cell-free solution comprising a set of enzymes and reagents selected from:
(a) a NAD kinase, NAD, and ATP;
(b) a NMN adenyltransferase, a NAD kinase, NMN and ATP; and/or
(c) a NAD kinase, ribose, nicotinate, NH⁴⁺ and ATP.

In at least one embodiment of the composition, the cell-free solution further comprises enzymes and/or reagents selected from: an NADP-dependent enzyme; a substrate for the NADP-dependent enzyme; ATP; and a combination thereof.

In at least one embodiment of the composition, the NAD kinase is selected from an exemplary NAD kinase of Table 2, such as EcNadK (WP_001059169.1), GsppnK1 (WP_011232266.1), GsppnK2 (WP_011230331.1), BliNadK (WP_009329373.1), TkNadK (WP_011251074.1), TmNadK (WP_004082250.1), PhNadK (WP_010885160.1), AfNadK (WP_010879860.1), CsNadK (WP_009610251.1), AtNadK (WP_003518725.1), ToNadK (WP_012572594.1), UTR1 (NP_012583.1), MtNadK (WP_003408383.1), BsNadK (WP_106610845.1), or a mixture thereof.

In at least one embodiment of the composition, the NMN adenyltransferase is selected from: an NMN adenyltransferase of Table 3, such as ScNma1 (NP_013432.1), ScNma2 (NP_011524.1), POF1 (NP_009883.1), RnNmnat2 (NP_001041507.1), BtNmnat1 (NP_001069302.1), BtNmnat2 (XP_005217345.1), MmNmnat1 (NP_001343286.1), MmNmnat2 (NP_780669.1), MmNmnat3 (NP_653116.1), HsNmnat1 (NP_001284707.1), HsNmnat2 (NP_055854.1), HsNmnat3 (NP_001307440.1), AtNmnat (NP_200392.3), or a mixture thereof.

In at least one embodiment of the composition, the cell-free solution further comprises the enzymes: RbsK, Prs, PncB, NadD, and NadE; optionally, wherein the enzymes are GsRbsK (ALA70174.1), TkPrs (WP_011251185.1), EcPncB (WP_001307697.1), GsNadD (WP_053532206.1), and EcNadE (EFI2440128.1).

In at least one embodiment of the composition, the cell-free solution can further comprise: (a) a precursor substrate, and one or more enzymes capable of converting the precursor substrate to the substrate for the NADP-dependent enzyme; and/or (b) one or more enzymes capable of converting the precursor of the compound produced by the NADP-dependent enzyme to the compound.

In another embodiment, the present disclosure also provides a process for cell-free production of NADP comprising contacting in a cell-free solution under suitable reaction conditions a set of enzymes and reagents selected from:
(a) a NAD kinase, NAD, and ATP;
(b) a NMN adenyltransferase, a NAD kinase, NMN and ATP; and/or
(c) a NAD kinase, ribose, nicotinate, NH⁴⁺ and ATP.

In at least one embodiment of the process for cell-free production of NADP, the cell-free solution does not comprise NADP or NADPH prior to contacting with (a) NAD, and a NAD kinase; (b) NMN, a NMN adenyltransferase, and a NAD kinase; and/or (c) ribose, nicotinate, NH⁴⁺, and a NAD kinase.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts a schematic summary of an exemplary biosynthetic pathway for the production of isobutanol that includes a “molecular rheostat” as described in US patent number U.S. Pat. No. 10,760,103B2.

FIG. 2A depicts a schematic summary of an exemplary biosynthetic pathway for the production of NADP. The pathway requires the substrates ribose (or xylose), NH⁴⁺, ATP, and nicotinamide, and uses the enzymes NAMPT and NMNAT to proceed from nicotinamide, through the NADP precursors, NMN to NAD, as described in the present disclosure. The final step the utilizes the enzyme, NAD kinase (NadK) to convert NAD to NADP.

FIG. 2B depicts a schematic summary of an exemplary biosynthetic pathway for the production of NADP. The pathway requires the substrates ribose (or xylose), NH⁴⁺, ATP, and nicotinate, and uses the enzymes PncB, NadD, and NadE to proceed from nicotinate, through the NADP precursors, NaMN, NaAD, to NAD, as described in the present disclosure. The final step the utilizes the enzyme, NAD kinase (NadK) to convert NAD to NADP.

FIG. 3A depicts a schematic summary of the final step in the biosynthetic pathway that utilizes the NAD kinase, EcNadK to convert NAD to NADP. The schematic illustrates how the production of NADP can be monitored via the FDH-catalyzed conversion of formate to CO₂ using the enzyme BsFDH which naturally prefers the substrate NADP as a coenzyme for this oxidation reaction. The concomitant conversion of NADP to NADPH is monitored at 340 nm.

FIG. 3B depicts a plot of exemplary assay results (plot of absorption at 340 nm over time) showing the enzymatic production of NADP from NAD (2 mM) via the activity of the NadK enzyme EcNadK (12 μg added) and the concomitant conversion of NADP to NADPH by the FDH enzyme BsFDH (50 μg added), as described in Example 2.

FIG. 4A depicts a schematic summary of the final step in the biosynthetic pathway that utilizes the NMN adenyltransferase, ScNma1, and the NAD kinase, EcNadK to convert NMN to NAD and NAD to NADP. The schematic illustrates how the production of NADP can be monitored via the FDH-catalyzed conversion of formate to CO₂ using the enzyme BsFDH which naturally prefers the substrate NADP as a coenzyme for this oxidation reaction. The concomitant conversion of NADP to NADPH is monitored at 340 nm.

FIG. 4B depicts a schematic summary of the step in the biosynthetic pathway that utilizes the NMN adenyltransferase, ScNma1 to convert NMN to NAD. The schematic illustrates how the production of NAD can be monitored via the activity of the enzyme, GsZwf which converts the added Glucose-6-phosphate (G6P) substrate to the product 6PGDL. The concomitant conversion of NAD to NADH is monitored at 340 nm.

FIG. 4C depicts a plot of exemplary assay results (plot of absorption at 340 nm over time) showing the enzymatic production of 6PGDL from NMN, in the presence of G6P and the enzymes ScNma1 and GsZwf (as described in Example 3), thereby indicating that ScNma1 is capable of converting NMN+ to NAD+.

FIG. 5 depicts a plot exemplary assay results (plot of absorption at 340 nm over time) showing the enzymatic production of NADP from NMN, via the activity of the enzymes ScNma1 and EcNadK (as described in Example 3). The conversion of NADP to NADPH by the FDH enzyme BsFDH is monitored at 340 nm. BsFdh prefers to use NADP as a coenzyme to oxidize formate to form CO₂.

FIG. 6A depicts a schematic summary of a biosynthetic pathway that utilizes the combination of five enzymes, Rbsk, Prs, PncB, NadD, and NadE, to carry out the in situ convert the substrates ribose, nicotinate, ammonia (NH₄⁺), and ATP to the coenzyme, NAD. NAD production can be monitored by the production of NADH in the presence of the substrate G6P and the enzyme, GsZwf. The addition of the enzyme, NadK, to this five enzyme pathway for the in situ production of NAD, further allows for the in situ production of NADP that can be used in a cell-free production of isobutanol (as described in Example 4).

FIG. 6B depicts a plot of exemplary assay results (plot of absorption at 340 nm over time) showing the enzymatic production of 6PGDL in the presence of the substrates, ribose, nicotinate, ATP, NH⁴⁺, G6P, and the six enzymes GsRbsK, TkPrs, EcPncB, GsNadD, EcNadE, and EcNadK (as described in Example 4).

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### DEFINITIONS

“Conversion” as used herein refers to the enzymatic conversion of the substrate(s) to the corresponding product(s). “Percent conversion” refers to the percent of the substrate that is converted to the product within a period of time under specified conditions. Thus, the “enzymatic activity” or “activity” of an enzymatic conversion can be expressed as “percent conversion” of the substrate to the product.

“Product” as used herein in the context of an enzyme mediated process refers to the compound or molecule resulting from the activity of the enzyme.

“Substrate” as used herein in the context of an enzyme mediated process refers to the compound or molecule acted on by the enzyme . . .

“NADP-dependent enzyme” as used herein refers to an enzyme that uses nicotinamide adenine dinucleotide phosphate (NADP) as a coenzyme for the conversion of a substrate to a product.

“NADP precursor compound” or “NADP precursor substrate” as used herein refers a compound or molecule acted on by an enzyme in a biosynthetic step for producing NADP. Exemplary NADP precursor compounds are provided in Table 1.

TABLE 1
Exemplary NADP precursor substrate compounds

| Compound Name | Abbrev. |
|---|---|
| nicotinamide | NA |
| β-nicotinamide D-ribonucleotide | NMN |
| nicotinamide adenine dinucleotide | NAD |
| nicotinate | NaA |
| β-nicotinate D-ribonucleotide | NaNMN |
| nicotinate adenine dinucleotide | NaAD |
| ribose | Rib |
| xylose | Xyl |
| D-ribose 5-phosphate | R5P |
| 5-phospho-α-D-ribose 1-diphosphate | PRPP |

“NAD kinase” or “NADK” as used herein refers to an enzyme that phosphorylates nicotinamide adenine dinucleotide (NAD+) to produce the coenzyme, NAD and includes those enzymes of classification EC 2.7.1.23. Exemplary NAD kinases useful in the compositions and methods of the present disclosure include but are not limited to those enzymes listed in Table 2 below and the accompanying Sequence Listing.

TABLE 2
Exemplary NAD kinases (NadK)

| | | | SEQ |
|---|---|---|---|
| | | | ID |
| NadK | Organism | Accession # | NO: |
| EcNadK | Escherichia coli | WP_001059169.1 | 1 |
| GsppnK1 | Geobacillus stearothermophilus | WP_011232266.1 | 2 |
| GsppnK2 | Geobacillus stearothermophilus | WP_011230331.1 | 3 |
| BliNadK | bacillus licheniformis | WP_009329373.1 | 4 |
| TkNadK | Thermococcus kodakarensis | WP_011251074.1 | 5 |
| TmNadK | Thermotoga maritima | WP_004082250.1 | 6 |
| PhNadK | Pyrococcus horikoshii | WP_010885160.1 | 7 |
| AfNadK | Archaeoglobus fulgidus | WP_010879860.1 | 8 |
| CsNadK | Caldanaerobacter subterraneus | WP_009610251.1 | 9 |
| AtNadK | Acetivibrio thermocellus | WP_003518725.1 | 10 |
| ToNadK | Thermococcus onnurineus | WP_012572594.1 | 11 |
| UTR1 | Saccharomyces cerevisiae | NP_012583.1 | 12 |
| MtNadK | Mycobacterium tuberculosis | WP_003408383.1 | 13 |
| BsNadK | Bacillus subtilis | WP_106610845.1 | 14 |

Additionally, it is also contemplated that NAD kinases useful in the compositions and processes of the present disclosure can include functional variants of the NAD kinases of Table 2 having at least 90%, at least 91%, at least 92%, at least 93%, at least 94%, at least 95%, at least 96%, at least 97%, at least 98%, or at least 99% amino acid sequence identity to an amino acid sequence of a NAD kinase listed in Table 2 and sufficient NAD kinase activity. It is contemplated that such NAD kinases can include, but are not limited to, any known NAD kinase functional variant or homolog having sufficient NAD kinase activity, any engineered versions of the NAD kinases listed in Table 2, and/or functional fragments or fusions of the NAD kinases listed in Table 2. Engineered versions of the NAD kinases listed in Table 2 can include, for example, NAD kinases with from 1-20 conservative amino acid substitutions relative to the enzymes listed in Table 2 that have sufficient NAD kinase activity. Functional fragments of the NAD kinases listed in Table 2, such as a fragment having from 1-20 amino acids truncated from the N-terminus or C-terminus that have sufficient NAD kinase activity. Functional fusions of the NAD kinases listed in Table 2 include but are not limited to His-tagged versions of the enzymes listed in Table 2. Techniques for preparing such engineered versions, functional fragments, and fusions of enzymes, such as NAD kinase, are well known to those of ordinary skill in the art.

“NMN adenyltransferase” or “NMNAT” as used herein refers to a nicotinamide-nucleotide adenyltransferase of enzyme classification EC 2.7.7.1) that catalyzes the conversion of the two substrates ATP and nicotinamide mononucleotide (NMN), to the two products are diphosphate and NAD+. Exemplary NMNAT enzymes useful in the compositions and methods of the present disclosure are listed in Table 3 below and the accompanying Sequence Listing.

TABLE 3
Exemplary NMN adenyltransferases (NMNAT)

| | | | SEQ ID |
|---|---|---|---|
| NMNAT | Organism | Accession number | NO: |
| POF1 | Saccharomyces cerevisiae | NP_009883.1 | 15 |
| ScNma1 | Saccharomyces cerevisiae | NP_013432.1 | 16 |
| ScNma2 | Saccharomyces cerevisiae | NP_011524.1 | 17 |
| RnNmnat2 | Rattus norvegicus | NP_001041507.1 | 18 |
| BtNmnat1 | Bos taurus | NP_001069302.1 | 19 |
| BtNmnat2 | Bos taurus | XP_005217345.1 | 20 |
| MmNmnat1 | Mus musculus | NP_001343286.1 | 21 |
| MmNmnat2 | Mus musculus | NP_780669.1 | 22 |
| MmNmnat3 | Mus musculus | NP_653116.1 | 23 |
| HsNmnat1 | Homo sapiens | NP_001284707.1 | 24 |
| HsNmnat2 | Homo sapiens | NP_055854.1 | 25 |
| HsNmnat3 | Homo sapiens | NP_001307440.1 | 26 |
| AtNmnat | Arabidopsis thaliana | NP_200392.3 | 27 |

Additionally, it is also contemplated that NMN adenyltransferases useful in the compositions and processes of the present disclosure can include functional variants of the NMN adenyltransferases of Table 3 having at least 90%, at least 91%, at least 92%, at least 93%, at least 94%, at least 95%, at least 96%, at least 97%, at least 98%, or at least 99% amino acid sequence identity to an amino acid sequence of a NMN adenyltransferases listed in Table 3 and sufficient NMN adenyltransferase activity. It is contemplated that such NMN adenyltransferases can include, but are not limited to, any known NMN adenyltransferase variant or homolog having sufficient NMN adenyltransferase activity, any engineered versions of the NMN adenyltransferases listed in Table 3, and/or functional fragments or fusions of the NMN adenyltransferases listed in Table 3. Engineered versions of the NMN adenyltransferases listed in Table 3 can include, for example, NMN adenyltransferases with from 1-20 conservative amino acid substitutions relative to the enzymes listed in Table 3 that have sufficient NMN adenyltransferase activity. Functional fragments of the NMN adenyltransferases listed in Table 3, such as a fragment having from 1-20 amino acids truncated from the N-terminus or C-terminus that have sufficient NMN adenyltransferase activity. Functional fusions of the NMN adenyltransferases listed in Table 3 include but are not limited to His-tagged versions of the enzymes listed in Table 3. Techniques for preparing such engineered versions and functional fragments and fusions of enzymes, such as NMN adenyltransferase, are well known to those of ordinary skill in the art.

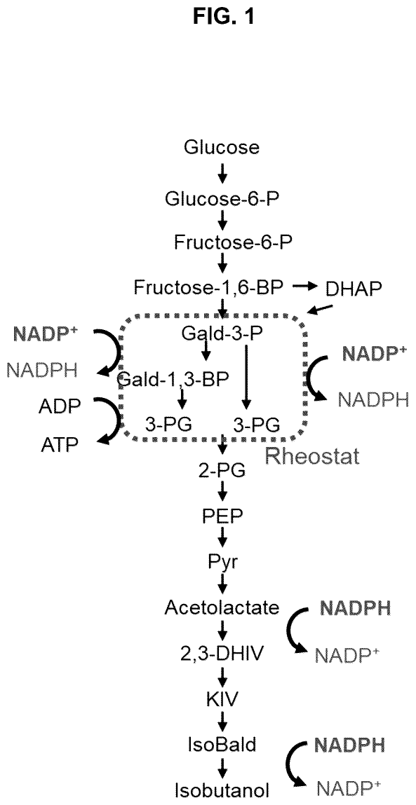

A set of enzymes are used in the cell-free systems for the biosynthesis of isobutanol using the process depicted in the scheme of FIG. 1. These enzymes are described in greater detail in U.S. Pat. No. 10,760,103B2, and in Sherkhanov et al. (“Isobutanol production freed from biological limits using synthetic biochemistry,” Nat Commun. 11, 4292 (2020)). It is contemplated that the sets of enzymes described in these references can also be used in the cell-free biosynthesis systems of the present disclosure that further include enzymes for in situ NADP production. The present disclosure provides sets of exemplary enzymes useful in such cell-free biosynthesis systems in the following examples. These exemplary enzymes are summarized in Table 4 below and the accompanying Sequence Listing.

TABLE 4
Enzymes used in cell-free isobutanol biosynthesis examples

| | | Source | | SEQ ID |
|---|---|---|---|---|
| Abbrev | Enzyme Type | Organism | Accession# | NO: |
| TmHex | Hexokinase | T. maritima | AAD36537.1 | 28 |
| TmPgi | Phosphoglucoisomerase | T. maritima | WP_004081585.1 | 29 |
| EcPfkB | Phosphoglucokinase | E. coli | EFJ64522.1 | 30 |
| GsFBA | Fructose 1,6 biphosphate | G. stearo- | KOR93738.1 | 31 |
| | aldolase | thermophilus | | |
| TmTPI | Triose phosphate isomerase | T. maritima | AAA67520.1 | 32 |
| TkGapN | Non-phosphorylating | T. kodakarensis | WP_011249656.1 | 33 |
| | glyceraldehyde 3 phosphate | | | |
| | dehydrogenase | | | |
| GsGapD | Phosphorylating | G. stearo- | KOR95271.1 | 34 |
| HM6 | glyceraldehyde 3 phosphate | thermophilus | | |
| | dehydrogenase | | | |
| TmPGK | Phosphoglycerate kinase | T. maritima | AAD35771.1 | 35 |
| TtdgPGM | Phosphoglycerate mutase | T. thermophilus | WP_011026502.1 | 36 |
| GsEno | Phosphoenolpyruvate | G. stearo- | KOR95275 | 37 |
| | hydratase | thermophilus | | |
| TtPyk | Pyruvate kinase | T. thermophilus | WP_011227631.1 | 38 |
| BsAlsS | Acetolactate synthase | B. subtilis | WP_003244057.1 | 39 |
| GsllvC | Ketol-acid reductoisomerase | G. stearo- | WP_033014337.1 | 40 |
| | | thermophilus | | |
| SmllvD | Dihydroxy-acid dehydratase | S. mutans | ESS16062.1 | 41 |
| KivD | Keto-isovalerate | L. lactis | WP_085624856.1 | 42 |
| | decarboxylase | | | |
| EcYahK | Alcohol dehydrogenase | E. coli | WP_128424329.1 | 43 |
| GsRbsK | Ribokinase | G. stearo- | ALA70174.1 | 44 |
| | | thermophilus | | |
| TkPrs | Ribose-phosphate | T. kodakarensis | WP_011251185.1 | 45 |
| | diphosphokinase | | | |
| EcPncB | Nicotinate | E. coli | WP_001307697.1 | 46 |
| | phosphoribosyltransferase | | | |
| GsNadD | Nicotinate-nucleotide | G. stearo- | WP_053532206.1 | 47 |
| | adenylyltransferase | thermophilus | | |
| EcNadE | NH3-dependent NAD+ | E. coli | EFI2440128.1 | 48 |
| | synthetase | | | |
| NAMPT | Nicotinamide | Mus musculus | NP_067499.2 | 49 |
| | phosphoribosyltransferase | | | |
| Ppk | Polyphosphate kinase | Paenarthrobacter | ABM08865.1 | 50 |
| | | aurescens | | |

“Host cell” as used herein refers to a cell capable of being functionally modified with recombinant nucleic acids and functioning to express recombinant products, including polypeptides and compounds produced by activity of the polypeptides.

“Nucleic acid,” or “polynucleotide” as used herein interchangeably to refer to two or more nucleosides that are covalently linked together. The nucleic acid may be wholly comprised ribonucleosides (e.g., RNA), wholly comprised of 2′-deoxyribonucleotides (e.g., DNA) or mixtures of ribo- and 2′-deoxyribonucleosides. The nucleoside units of the nucleic acid can be linked together via phosphodiester linkages (e.g., as in naturally occurring nucleic acids), or the nucleic acid can include one or more non-natural linkages (e.g., phosphorothioester linkage). Nucleic acid or polynucleotide is intended to include single-stranded or double-stranded molecules, or molecules having both single-stranded regions and double-stranded regions. Nucleic acid or polynucleotide is intended to include molecules composed of the naturally occurring nucleobases (i.e., adenine, guanine, uracil, thymine and cytosine), or molecules comprising that include one or more modified and/or synthetic nucleobases, such as, for example, inosine, xanthine, hypoxanthine, etc.

“Protein,” “polypeptide,” and “peptide” are used herein interchangeably to denote a polymer of at least two amino acids covalently linked by an amide bond, regardless of length or post-translational modification (e.g., glycosylation, phosphorylation, lipidation, myristilation, ubiquitination, etc.). As used herein “protein” or “polypeptide” or “peptide” polymer can include D- and L-amino acids, and mixtures of D- and L-amino acids.

“Naturally-occurring” or “wild-type” as used herein refers to the form as found in nature. For example, a naturally occurring nucleic acid sequence is the sequence present in an organism that can be isolated from a source in nature and which has not been intentionally modified by human manipulation.

“Recombinant,” “engineered,” or “non-naturally occurring” when used herein with reference to, e.g., a cell, nucleic acid, or polypeptide, refers to a material, or a material corresponding to the natural or native form of the material, that has been modified in a manner that would not otherwise exist in nature, or is identical thereto but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Nucleic acid derived from” as used herein refers to a nucleic acid having a sequence at least substantially identical to a sequence of found in naturally in an organism. For example, cDNA molecules prepared by reverse transcription of mRNA isolated from an organism, or nucleic acid molecules prepared synthetically to have a sequence at least substantially identical to, or which hybridizes to a sequence at least substantially identical to a nucleic sequence found in an organism.

“Coding sequence” refers to that portion of a nucleic acid (e.g., a gene) that encodes an amino acid sequence of a protein.

“Heterologous nucleic acid” as used herein refers to any polynucleotide that is introduced into a host cell by laboratory techniques, and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the imine reductase enzymes may be codon optimized for optimal production from the host organism selected for expression.

“Preferred, optimal, high codon usage bias codons” refers to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; CodonW, John Peden, University of Nottingham; Mclnerney, J. O, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Control sequence” as used herein refers to all sequences, which are necessary or advantageous for the expression of a polynucleotide and/or polypeptide as used in the present disclosure. Each control sequence may be native or foreign to the nucleic acid sequence encoding a polypeptide. Such control sequences include, but are not limited to, a leader, a promoter, a polyadenylation sequence, a pro-peptide sequence, a signal peptide sequence, and a transcription terminator. At a minimum, control sequences typically include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” as used herein refers to a configuration in which a control sequence is appropriately placed (e.g., in a functional relationship) at a position relative to a polynucleotide sequence or polypeptide sequence of interest such that the control sequence directs or regulates the expression of the sequence of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Percentage of sequence identity,” “percent sequence identity,” “percentage homology,” or “percent homology” are used interchangeably herein to refer to values quantifying comparisons of the sequences of polynucleotides or polypeptides, and are determined by comparing two optimally aligned sequences over a comparison window, wherein the portion of the polynucleotide or polypeptide sequence in the comparison window may comprise additions or deletions (or gaps) as compared to the reference sequence for optimal alignment of the two sequences. The percentage values may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215:403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89: 10915). Exemplary determination of sequence alignment and % sequence identity can employ the BESTFIT or GAP programs in the GCG Wisconsin Software package (Accelrys, Madison Wis.), using default parameters provided.

“Reference sequence” refers to a defined sequence used as a basis for a sequence comparison. A reference sequence may be a subset of a larger sequence, for example, a segment of a full-length nucleic acid or polypeptide sequence. A reference sequence typically is at least 20 nucleotide or amino acid residue units in length, but can also be the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (or gaps) of 20 percent or less as compared to the reference sequence (which does not comprise additions or deletions) for optimal alignment of the two sequences.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered imine reductase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Isolated” as used herein in reference to a molecule means that the molecule (e.g., cannabinoid, polynucleotide, polypeptide) is substantially separated from other compounds that naturally accompany it, e.g., protein, lipids, and polynucleotides. The term embraces nucleic acids which have been removed or purified from their naturally-occurring environment or expression system (e.g., host cell or in vitro synthesis).

“Substantially pure” refers to a composition in which a desired molecule is the predominant species present (i.e., on a molar or weight basis it is more abundant than any other individual macromolecular species in the composition), and is generally a substantially purified composition when the object species comprises at least about 50 percent of the macromolecular species present by mole or % weight.

“Recovered” as used herein in relation to an enzyme, protein, or cannabinoid compound, refers to a more or less pure form of the enzyme, protein, or cannabinoid.

### In Situ NADP Production for Use in Cell-Free NADP-Dependent Biosynthesis Systems

NADP is an essential coenzyme used by NADP-dependent enzymes for the cell-free biosynthesis of biobased chemical compounds, such as isobutanol, 3-methyl-1-butanol, leucine, and valine. FIG. 1 depicts a schematic of an exemplary biosynthetic pathway for isobutanol production that is further described in U.S. Pat. No. 10,760,103B2, which is hereby incorporated by reference herein for all purposes. As shown in the scheme of FIG. 1, NADP acts as a redox carrier that transfers electrons generated by glycolysis to isobutyraldehyde for the production of isobutanol and maintains the redox balance of the entire system. NADP, however, is a relatively expensive reagent that adds significant costs affecting the commercial viability of any biobased chemical production using NADP-dependent enzymes. The present disclosure describes various alternative sets of enzymes (see e.g., Tables 2 and 3) and NADP precursor substrates (see e.g., Table 1) that can be used in cell-free solutions to biosynthesize NADP in situ. This in situ produced NADP can then be used by NADP-dependent enzymes also present in the solution. Accordingly, the present disclosure includes cell-free solution compositions containing sets of reagents and enzymes, as well as processes of using these composition in cell-free biosynthesis systems that require NADP, such as the isobutanol production system of FIG. 1.

As described elsewhere herein, including the Examples, the present disclosure provides a cell-free process, and associated compositions, useful for NADP-dependent biosynthesis of a biobased chemical compound, such as isobutanol, 3-methyl-1-butanol, leucine, or valine. The cell-free processes, and associated compositions, include a combination of reagents and enzymes that are capable of in situ production of NADP, which can then be used to fuel NADP-dependent enzymes used in a biosynthetic process, such as an isobutanol production process as depicted in FIG. 1. Generally, the processes comprise contacting a cell-free solution composition that contains a set of enzymes and NADP precursor reagents, under suitable reaction conditions with the NADP-dependent enzyme, the substrate for the NADP-dependent enzyme, and ATP. Notably, there is no need for the coenzyme NADP in this cell-free solution containing the enzymes and reagents. The NADP-dependent enzyme uses NADP produced in situ to convert its substrate to the desired product. That is, the NADP is produced in the solution and used by the NADP-dependent enzyme in the conversion of the substrate to the compound or to a precursor of the compound.

The in situ production of NADP is carried out via enzymatic conversion of a NADP precursor compound such as NAD, NMN, and/or nicotinate and ribose (or xylose). Exemplary combinations of NADP precursor compounds and enzymes useful in the compositions and processes of the present disclosure for the in situ biosynthesis of NADP include: (a) NAD, and a NAD kinase; (b) NMN, a NMN adenyltransferase, and NadK; and/or (c) ribose, nicotinate, NH⁴⁺, and a NAD kinase. The use of one or more of these combinations of enzymes and NADP precursor compounds in a cell-free solution system for the in situ production of NADP is demonstrated and further described in the Examples.

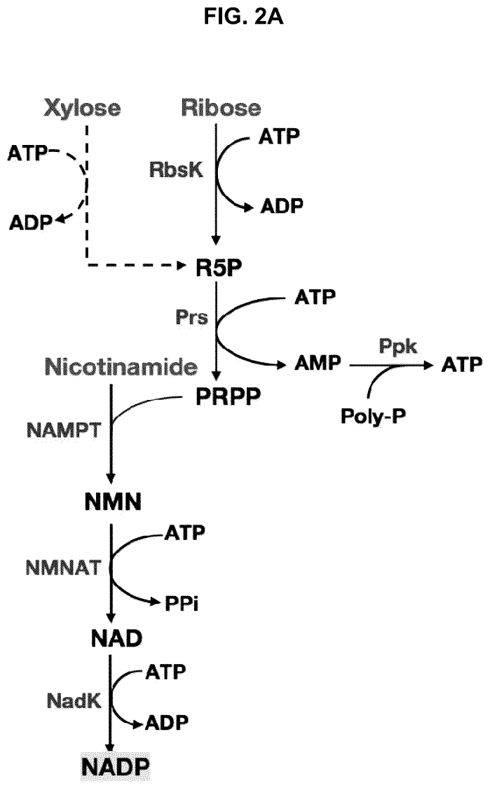

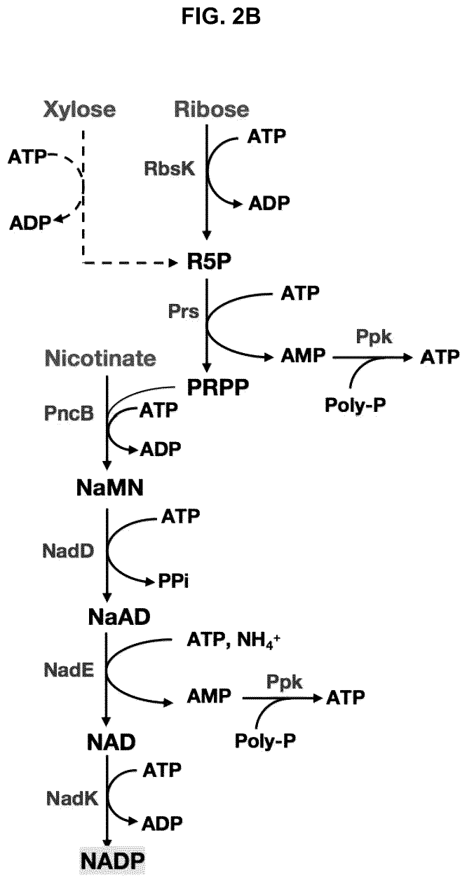

FIG. 2A depicts an exemplary biosynthetic pathway for in situ production of NADP that can be incorporated into other cell-free biosynthesis systems, such as isobutanol production. As shown in FIG. 2A, the pathway requires input of the substrates ribose (or xylose), NH⁴⁺, ATP, and nicotinamide, and then uses the enzymes NAMPT and NMNAT to proceed from nicotinamide, through the NADP precursors, NMN to NAD, to the final step producing NADP. This final step utilizes the enzyme, NAD kinase (NadK) to convert NAD to NADP. FIG. 2B depicts an alternative to the pathway of FIG. 2A that utilizes nicotinate rather than nicotinamide as the input substrate leading to NADP. This pathway also requires the substrates ribose (or xylose), NH⁴⁺, and ATP, but uses the enzymes PncB, NadD, and NadE to proceed from nicotinate, through the NADP precursors, NaMN, NaAD, to NAD. As in the pathway of FIG. 2A, the final step the utilizes the enzyme, NAD kinase (NadK) to convert NAD to NADP.

Accordingly, in at least one embodiment, the present disclosure includes a cell-free process for in situ NADP-dependent biosynthesis of a compound (e.g., isobutanol), where the biosynthesis process comprises contacting the cell-free solution in which the process is occurring with ribose, nicotinate, NH⁴⁺, and a NAD kinase (NadK) under suitable reaction conditions. Typically, the cell-free solution would also include ATP, one or more NADP-dependent enzymes, and one or more substrates for the NADP-dependent enzymes used in the process. For example, exemplary enzymes also included in the cell-free solution for production of isobutanol as shown in Table 4, and described in the Examples. As shown in FIG. 2B, in addition to NadK, the pathway that converts nicotinate to NADP also requires the enzymes, PncB, NadD, and NadE. These enzymes are often also present in the cell-free solution because they are commonly involved in other biosynthesis steps involved in the production of the biobased target compound, As demonstrated in the Examples of the present disclosure, the mixture of enzymes in the cell-free solutions used in the biosynthetic production of isobutanol (e.g., as in FIG. 1) also include PncB, NadD, and NadE.

As noted above, FIG. 2A requires added nicotinamide and FIG. 2B requires added nicotinate. A common feature of the pathways, however, is the final step that utilizes the NadK to convert the precursor, NAD, to the desire coenzyme product NADP. The present disclosure contemplates cell-free systems that short-circuit the pathways of FIG. 2A and FIG. 2B by utilizing the added precursor substrate, NAD, for the in situ production of NADP. Accordingly, in at least one embodiment, the present disclosure includes a cell-free process for in situ NADP-dependent biosynthesis of a compound (e.g., isobutanol), where the biosynthesis process comprises contacting the cell-free solution in which the process is occurring with NAD, and a NAD kinase under suitable reaction conditions. Typically, the cell-free solution would also include ATP, one or more NADP-dependent enzymes, and one or more substrates for the NADP-dependent enzymes used in the process. For example, exemplary enzymes also included in the cell-free solution for production of isobutanol as shown in Table 4, and described in the Examples. NadK enzymes useful in such a process for in situ production of NADP by conversion of NAD are provided in Table 2. In at least one embodiment, the NadK used in such a process to convert NAD to NADP is EcNadK (WP_001059169.1). Typical reaction conditions for carrying out this enzymatic reaction are provided in the Examples and known in the art.

As shown in FIG. 2A, the precursor substrate NAD is produced by the conversion of the precursor substrate, NMN, which is catalyzed by the enzyme NMN adenyltransferase. Accordingly, in at least one embodiment, the present disclosure also includes a cell-free process for in situ NADP-dependent biosynthesis of a compound (e.g., isobutanol), where the biosynthesis process comprises contacting the cell-free solution in which the process is occurring with the precursor substrate NMN, a NMN adenyltransferase (NMNAT), and a NAD kinase under suitable reaction conditions. This pathway starting with the added precursor substrate, NMN, and the enzymes NMNAT and NadK, also results in the in situ production of NADP. As described above and elsewhere herein, this NADP could then be used by other NADP-dependent enzymes also present in the solution as part of a cell-free process, such as production of isobutanol. Thus, the cell-free solution would also include ATP, one or more NADP-dependent enzymes, and one or more substrates for the NADP-dependent enzymes used in the process. For example, exemplary enzymes also included in the cell-free solution for production of isobutanol as shown in Table 4, and described in the Examples. NMNAT enzymes useful in such a process for in situ production of NADP by conversion of NAD are provided in Table 3. In at least one embodiment, the NMNAT used in such a process to convert NMN to NADP is ScNma1 (NP_013432.1). Typical reaction conditions for carrying out this NMNAT-catalyzed reaction are provided in the Examples and known in the art.

Thus, using the combinations of NADP precursor compounds and enzymes as disclosed herein, a cell-free solution that uses an NADP-dependent enzyme for the biosynthesis of a biobased compound (e.g., isobutanol, 3-methyl-1-butanol, leucine, or valine) does not need NADP or NADPH added as a reagent, as the required NADP is produced in situ by the activity of the added enzymes on the NADP precursor compounds (e.g., nicotinate, NAD, and/or NMN). Accordingly, a cell-free solution for NADP-dependent biosynthesis of a compound need not include any NADP or NADPH prior to contacting the solution with an enzyme and NADP precursor reagent combination of the present disclosure that results in production of NADP in situ. Those combinations of enzymes and NADP precursor reagents can include, but are not limited to, the following: (a) NAD, and a NAD kinase; (b) NMN, a NMN adenyltransferase, and a NAD kinase; and/or (c) ribose, nicotinate, NH⁴⁺, and a NAD kinase.

For example, a cell-free process for NADP-dependent biosynthesis of a compound (e.g., isopropanol) can be carried out by contacting in a cell-free solution under suitable reaction conditions: an NADP-dependent enzyme; a substrate for the NADP-dependent enzyme; ATP; and a combination of reagents and enzymes selected from: (a) NAD, and a NAD kinase; (b) NMN, a NMN adenyltransferase, and a NAD kinase; and/or (c) ribose, nicotinate, NH⁴⁺, and a NAD kinase. It is not necessary to add NADP to the reaction mixture, and the necessary NADP is produced in the solution where it can be used by the NADP-dependent enzyme also present in the solution for the conversion of the substrate to the compound or to a precursor of the compound. That is, the cell-free solution does not need to include any added NADP or NADPH prior to contacting with any of the NADP producing enzyme and reagent combinations.

It is contemplated that processes and compositions comprising the combinations of enzymes and NADP precursor reagents of the present disclosure can be incorporated (or “dropped in”) to any cell free biosynthetic process that utilizes an NADP dependent enzyme simply by adding the enzymes and reagents to the reaction mixture. A range of such cell-free biosynthetic processes, including suitable sets of enzymes, substrates, reagents, and reaction conditions, for production of compounds such as isobutanol, 3-methyl-1-butanol, leucine, and valine are known in the art. For example, a cell-free isobutanol biosynthesis system is disclosed in U.S. Pat. No. 10,760,103 B2, which is hereby incorporated herein by reference. Further details of including suitable sets of enzymes, substrates, reagents, and reaction conditions for carrying out such cell-free biosynthesis processes are disclosed in the Examples.

Generally, the cell free solutions comprising a combination of reagents and enzymes of the present disclosure can include additional reagents and enzymes that may be required to carry out the particular NADP-dependent biosynthesis reaction. For example, the cell-free solution can further comprise a precursor substrate that can be converted to the substrate for the NADP-dependent enzyme, and additionally an enzyme capable of converting that precursor substrate to the desired substrate. In at least one embodiment, the cell-free solution can include a precursor substrate that is a sugar, such as a C6 sugar (e.g., xylose), a C5 sugar (e.g., ribose), or a mixture of a C6 and C5 sugar. Various pathways of enzymes leading from precursor substrates (e.g., sugars) to substrates that can be converted by an NADP dependent enzyme to a desired biobased chemical compound in a cell free process are known in the art.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Cell-Free Biosynthetic Production of Isobutanol Using NADP

This example illustrates that the added coenzyme nicotinamide adenine dinucleotide phosphate (NADP) functions as the sole redox carrier to maintain isobutanol production in the exemplary cell-free biosynthetic system.

### Materials and Methods

### EM7 Mixture

The EM7 reaction mixture used for the cell-free production of isobutanol in this example includes a composition of enzymes and substrates/reagents as shown in Table 5 (below).

TABLE 5
EM7 mixture

| | | |
|---|---|---|
| TmHex | 0.07 | mg/ml |
| TmPgi | 0.2 | mg/ml |
| EcPfkB | 0.2 | mg/ml |
| GsFba | 0.35 | mg/ml |
| TmTpi | 0.07 | mg/ml |
| TkGapN | 0.46 | mg/ml |
| AfGapDH | 0.05 | mg/ml |
| TmPgk | 0.1 | mg/ml |
| TtdgPgm | 0.18 | mg/ml |
| GsEno | 0.3 | mg/ml |
| TtPyk | 0.15 | mg/ml |
| BsAlsS | 0.15 | mg/ml |
| GsllvC | 1.7 | mg/ml |
| SmllvD | 0.9 | mg/ml |
| KivD | 0.07 | mg/ml |
| EcYahK | 0.27 | mg/ml |
| Bis-tris propane pH 8.5 | 100 | mM |
| KCl | 10 | mM |
| MgCl₂ | 12.5 | mM |
| Glutathione | 1 | mM |
| Glucose-1-phosphate | 0.75 | mM |
| TPP | 0.5 | mM |
| PO₄ | 5 | mM |
| ATP | 10 | mM |
| NADP⁺ | 2 to 6 | mM |
| NADPH | 0 to 6 | mM |

| | |
|---|---|
| Cellulosic hydrolysate | 30% |

The reactions are set at 32 °C for 16 h, and 2 mM NADP⁺ is used for isobutanol production. NADP to NADPH conversion is monitored at 340 nm through monitoring the activity of the formate dehydrogenase BsFDH (which prefers NADP⁺ as coenzyme) in conversion of formate to CO₂.

### Results

As shown by the results summarized in Table 6, the oxidized coenzyme, NADP⁺, can function as the sole redox carrier to maintain isobutanol production in a cell-free system. Addition of the reduced form, NADPH, was not needed to maintain isobutanol production.

TABLE 6

| Enzyme Mixture | EM7 | EM7 | EM7 | EM7 | EM7 | EM7 |
|---|---|---|---|---|---|---|
| Sugar | C6 | C6 | C6 | C6 | C6 | C6 |
| NADPH (mM) | 6 | 0 | 0 | 0 | 0 | 0 |
| NADP⁺ (mM) | 6 | 6 | 5 | 4 | 3 | 2 |
| Isobutanol (g/L) | 22.8 | 23.8 | 25.4 | 23.6 | 23.7 | 24.1 |

These results demonstrate a baseline cell-free system that uses only added NADP (without any added NADPH) to maintain isobutanol production.

### Example 2: Cell-Free Biosynthetic Production of Isobutanol Using NAD

This example illustrates a study showing that nicotinamide adenine dinucleotide (NAD) can function as the sole redox carrier to maintain isobutanol production in the cell-free biosynthetic system (with no added NADP). In the exemplified biosynthetic process, added NAD is converted in situ to NADP⁺ by the E. coli enzyme, NAD kinase (EcNadK) which is added to the cell-free isobutanol biosynthesis system. The final step of the biosynthetic reaction utilizes the NAD kinase, EcNadK to convert NAD⁺ to NADP⁺.

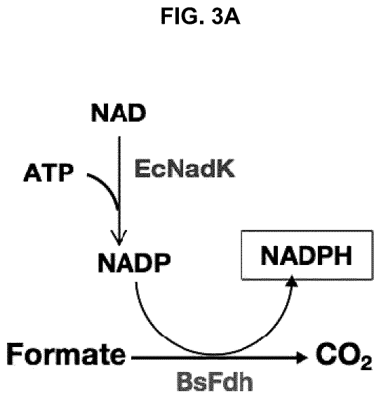

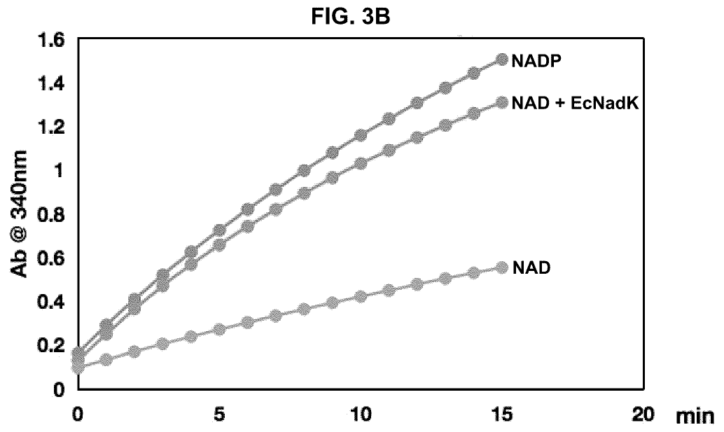

As shown in the scheme of FIG. 3A, NAD is converted to NADP by EcNadK. This production of NADP can be monitored using the formate dehydrogenase, BsFdh, which naturally prefers using NADP⁺ to oxidize formate to CO₂. This oxidation reaction results in the conversion of the NADP⁺ produced by EcNadK to NADPH with a concomitant increase in absorption at 340 nm.

### Materials and Methods

### Cell-Free Isobutanol Production Using EM15 or EM18 Reaction Mixtures

The EM15 reaction mixture used in this example replaces 0.05 mg/ml AfGapDH used in EM7 reaction mixture (described in Example 1) with 0.1 mg/ml GsGapDHM6. The composition of the EM15 reaction mixture includes 2 mM NADP⁺ and is summarized in Table 7 (below).

TABLE 7
EM15 mixture

| | | |
|---|---|---|
| TmHex | 0.07 | mg/ml |
| TmPgi | 0.2 | mg/ml |
| EcPfkB | 0.2 | mg/ml |
| GsFba | 0.35 | mg/ml |
| TmTpi | 0.07 | mg/ml |
| TkGapN | 0.46 | mg/ml |
| GsGapDHM6 | 0.1 | mg/ml |
| TmPgk | 0.1 | mg/ml |
| TtdgPgm | 0.18 | mg/ml |
| GsEno | 0.3 | mg/ml |
| TtPyk | 0.15 | mg/ml |
| BsAlsS | 0.15 | mg/ml |
| GsllvC | 1.7 | mg/ml |
| SmllvD | 0.9 | mg/ml |
| KivD | 0.07 | mg/ml |
| EcYahK | 0.27 | mg/ml |
| Bis-tris propane pH 8.5 | 100 | mM |
| KCl | 10 | mM |
| MgCl₂ | 12.5 | mM |
| Glutathione | 1 | mM |
| Glucose-1-phosphate | 0.75 | mM |
| TPP | 0.5 | mM |
| PO₄ | 5 | mM |
| ATP | 10 | mM |
| NADP⁺ | 2 | mM |

| | |
|---|---|
| Cellulosic hydrolysate | 30% |

The EM18 mixture replaces the 2 mM NADP⁺ used in the EM15 reaction mixture with EcNadK and 2 mM NAD⁺. The composition of the EM18 reaction mixture is summarized in Table 8 (below).

TABLE 8
EM18 mixture

| | | |
|---|---|---|
| TmHex | 0.07 | mg/ml |
| TmPgi | 0.2 | mg/ml |
| EcPfkB | 0.2 | mg/ml |
| GsFba | 0.35 | mg/ml |
| TmTpi | 0.07 | mg/ml |
| TkGapN | 0.46 | mg/ml |
| GsGapDHM6 | 0.1 | mg/ml |
| TmPgk | 0.1 | mg/ml |
| TtdgPgm | 0.18 | mg/ml |
| GsEno | 0.3 | mg/ml |
| TtPyk | 0.15 | mg/ml |
| BsAlsS | 0.15 | mg/ml |
| GsllvC | 1.7 | mg/ml |
| SmllvD | 0.9 | mg/ml |
| KivD | 0.07 | mg/ml |
| EcYahK | 0.27 | mg/ml |
| EcNadK | 0.1 to 0.2 | mg/ml |
| Bis-tris propane pH 8.5 | 100 | mM |
| KCl | 10 | mM |
| MgCl₂ | 12.5 | mM |
| Glutathione | 1 | mM |
| Glucose-1-phosphate | 0.75 | mM |
| TPP | 0.5 | mM |
| PO₄ | 5 | mM |
| ATP | 10 | mM |
| NAD⁺ | 2 | mM |

| | |
|---|---|
| Cellulosic hydrolysate | 30% |

The cell free isobutanol reactions are set at 32 °C for 16 h, and NADP to NADPH conversion is monitored at 340 nm through the activity of formate dehydrogenase, BsFdh (50 μg) and added formate.

### Results

FIG. 3B depicts an exemplary plot of absorption at 340 nm over time showing production of NADPH in the presence of 2 mM NAD, 12 μg EcNadK, and 50 μg BsFDH without any added NADP is comparable to NADPH production with added NADP.

As shown by the results with the EM18 reaction mixture summarized in Table 9, the oxidized coenzyme, NAD⁺, can be converted by EcNadK in situ to produce NADP⁺ which can then function as the sole redox carrier to maintain isobutanol production in the EM18 cell-free system comparable to (and slightly better than) the isobutanol production by the EM15 cell-free system which uses added NADP.

TABLE 9

| Enzyme Mixture | EM15 | EM18 | EM18 | EM18 |
|---|---|---|---|---|
| Sugar | C6 | C6 | C6 | C6 |
| NADP⁺ (mM) | 2 | 0 | 0 | 0 |
| NAD⁺ (mM) | 0 | 2 | 2 | 2 |
| EcNadK (mg/mL) | 0 | 0.1 | 0.15 | 0.2 |
| Isobutanol (g/L) | 22.8 | 24.5 | 24.3 | 23.4 |

These results demonstrate that the combination of a NadK enzyme and an added NAD substrate can function as the sole redox carrier in a cell-free system to maintain isobutanol production without added NADP.

### Example 3: Cell-Free Biosynthetic Production of Isobutanol Using NMN

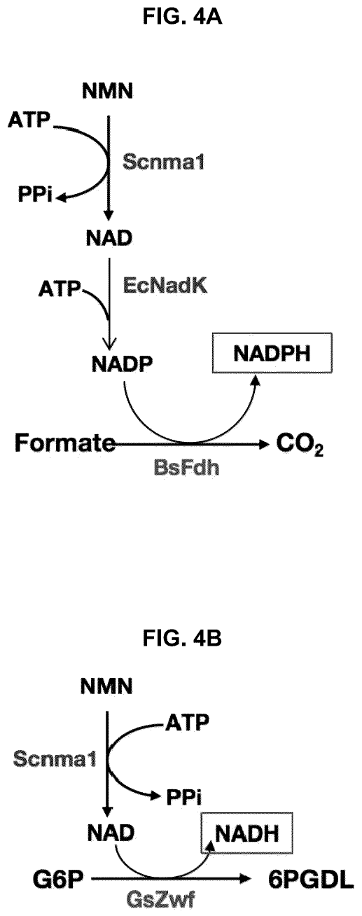

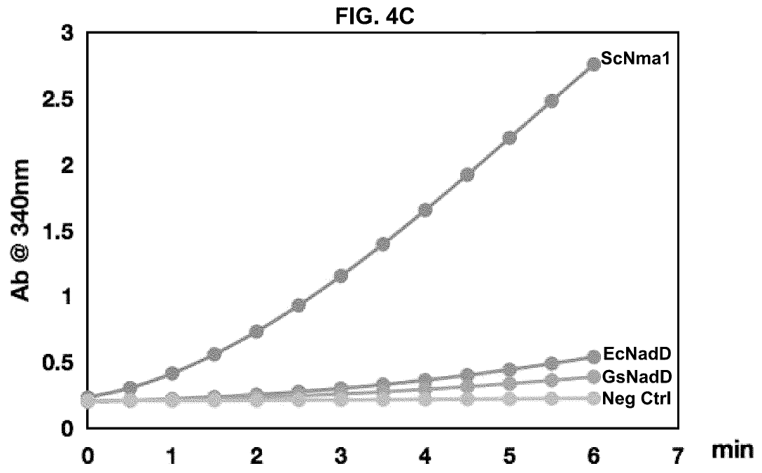

This example illustrates a study showing that β-nicotinamide D-ribonucleotide (NMN) can function as the sole redox carrier to maintain isobutanol production in a cell-free biosynthetic system (with no added NADP). In the exemplified biosynthetic process, added NMN⁺ is converted in situ to NADP⁺ by the enzymes, mononucleotide adenyltransferase (NMNAT) (ScNma1) and NAD kinase (EcNadK) which together can convert NMN to NADP in situ when added to the cell-free isobutanol biosynthesis system. This biosynthetic reaction is depicted in FIG. 4A. Further, as described in Example 2, the production of NADP by EcNadK in the final step is monitored using the formate dehydrogenase, BsFdh, which naturally prefers using NADP⁺ to oxidize formate to CO₂. This oxidation reaction results in the conversion of the NADP⁺ produced by EcNadK to NADPH with a concomitant increase in absorption at 340 nm.

### Materials and Methods

### Assay of ScNma1 Catalyzed Conversion of NMN to NAD

An initial enzyme assay was carried out to demonstrate that NMNAT enzyme, ScNma1 converts NMN⁺ to form NAD⁺ with NAD to NADH conversion monitored at 340 nm through the activity of the enzyme, GsZwf and added Glucose-6-phosphate (G6P) substrate, as summarized in the scheme of FIG. 4B.

FIG. 4C depicts an exemplary plot of absorption at 340 nm over time showing production of 6PGDL in the presence of NMN, 12 μg ScNma1, G6P and GsZwf. This production of 6PGDL indicates that ScNma1 can convert NMN⁺ to form NAD⁺ as shown in the scheme of FIG. 4B.

### Cell-Free Production of Isobutanol Using EM20 Mixture

The EM20 reaction mixture uses EcNadK and ScNma1 to biosynthesize NADP⁺ from NMN⁺ and ATP. The composition of the EM20 reaction mixture is summarized in Table 10 (below).

TABLE 10
EM20 mixture

| | | |
|---|---|---|
| TmHex | 0.07 | mg/ml |
| TmPgi | 0.2 | mg/ml |
| EcPfkB | 0.2 | mg/ml |
| GsFba | 0.35 | mg/ml |
| TmTpi | 0.07 | mg/ml |
| TkGapN | 0.46 | mg/ml |
| GsGapDHM6 | 0.1 | mg/ml |
| TmPgk | 0.1 | mg/ml |
| TtdgPgm | 0.18 | mg/ml |
| GsEno | 0.3 | mg/ml |
| TtPyk | 0.15 | mg/ml |
| BsAlsS | 0.15 | mg/ml |
| GsllvC | 1.7 | mg/ml |
| SmllvD | 0.9 | mg/ml |
| KivD | 0.07 | mg/ml |
| EcYahK | 0.27 | mg/ml |
| EcNadK | 0.1 | mg/ml |
| ScNma1 | 0.1 to 0.4 | mg/ml |
| Bis-tris propane pH 8.5 | 100 | mM |
| KCl | 10 | mM |
| MgCl₂ | 12.5 | mM |
| Glutathione | 1 | mM |
| Glucose-1-phosphate | 0.75 | mM |
| TPP | 0.5 | mM |
| PO₄ | 5 | mM |
| ATP | 10 | mM |
| NMN⁺ | 3 | mM |

| | |
|---|---|
| Cellulosic hydrolysate | 30% |

The EM15 and EM18 reaction mixtures were as described in Example 2. The reactions are set at 32 °C for 16 h, and 3 mM NMN⁺, 0.2 mg/mL ScNma1 and 0.1 mg/mL EcNadK is used for isobutanol production. NADP to NADPH conversion is monitored at 340 nm through the activity of formate dehydrogenase and added formate.

### Results

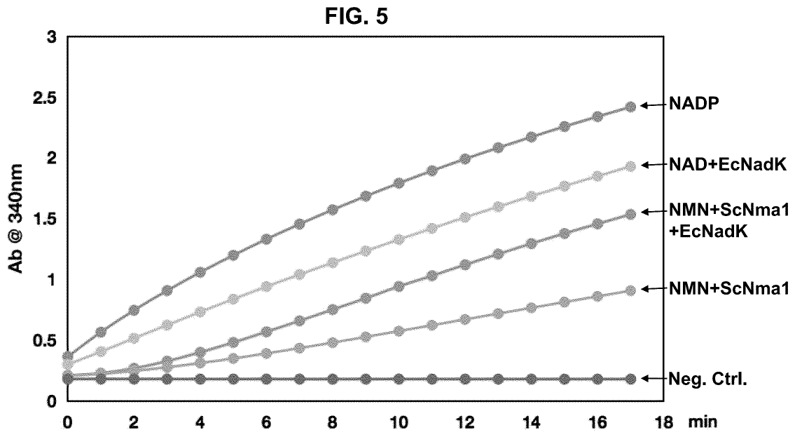

FIG. 5 depicts exemplary plots of absorption at 340 nm over time in the presence of 50 μg BsFDH showing production of NADPH in the presence of: (1) added NADP; (2) added NAD+EcNadK; (3) added NMN+ScNma1+EcNadK; (4) added NMN+ScNma1 with no added EcNadK; and (5) a negative control with no added NADP, NMN, or enzymes. The production rate of NADPH by the NMN+ScNma1+EcNadK system is comparable to the production by the added NAD⁺ EcNadK system.

As shown by the results for EM20 summarized in Table 11, NMN⁺ can be converted in situ to NAD⁺ by ScNma1, and the resulting NAD⁺ can be converted by EcNadK in situ to produce NADP⁺.

TABLE 11

| Enzyme Mixture | EM15 | EM18 | EM20 | EM20 | EM20 | EM20 |
|---|---|---|---|---|---|---|
| Sugar | C6 | C6 | C6 | C6 | C6 | C6 |
| NADP⁺ (mM) | 2 | 0 | 0 | 0 | 0 | 0 |
| NAD⁺ (mM) | 0 | 2 | 0 | 0 | 0 | 0 |
| NMN⁺ (mM) | 0 | 0 | 3 | 3 | 3 | 3 |
| EcNadK (mg/mL) | 0 | 0.1 | 0.1 | 0.1 | 0.1 | 0.1 |
| ScNma1 (mg/mL) | 0 | 0 | 0.1 | 0.2 | 0.3 | 0.4 |
| Isobutanol (g/L) | 21.3 | 21.95 | 21.35 | 21.85 | 22.45 | 22 |

These results demonstrate that the combination of an NMNAT and a NadK enzyme and a NMN substrate can function as the sole redox carrier in a cell-free system to maintain isobutanol production without added NADP.

### Example 4: Cell-Free Biosynthetic Production of Isobutanol Using NMN

This example illustrates a study showing that the coenzyme, NADP can be fully synthesized in situ from added nicotinate, ribose (or xylose), and ammonia using a series of enzymes. It is also demonstrated that this biosynthesized NADP can be used for isobutanol production in a cell-free biosynthesis system.

### Materials and Methods

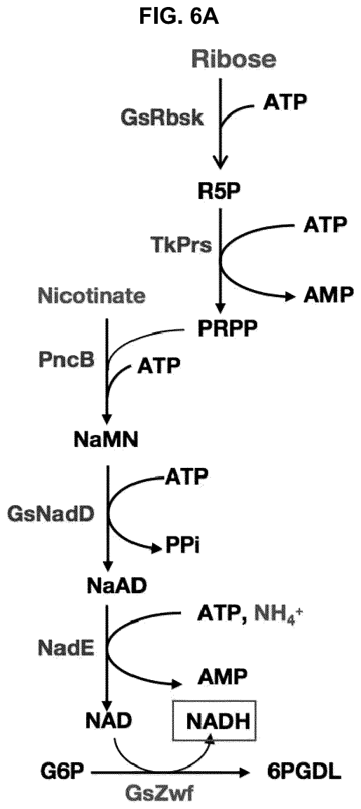

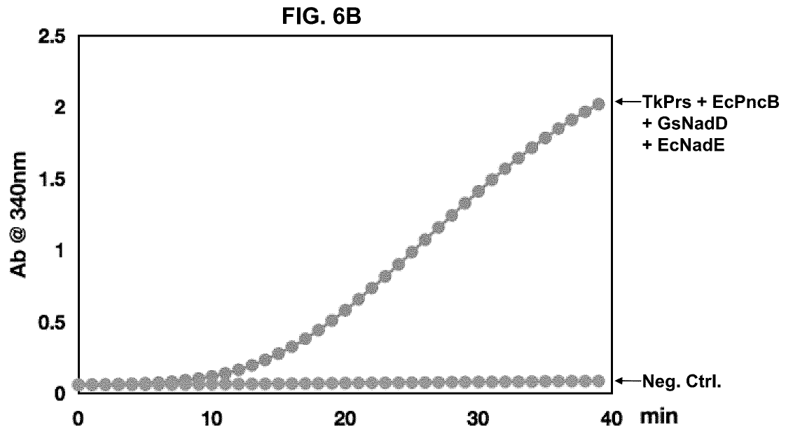

An initial assay was carried out to demonstrate that the substrates, ribose, nicotinate, NH₄⁺, and ATP, can be used to produce NAD in a biosynthetic reaction with NAD to NADH conversion monitored at 340 nm through the activity of the enzyme, GsZwf and added Glucose-6-phosphate (G6P) substrate (as in Example 3). The biosynthetic reaction pathway used in the assay is summarized in the scheme of FIG. 6A.

The amounts and relative thermostability of the enzymes used in biosynthetic reaction of the assay of FIG. 6A are shown in Table 12.

TABLE 12

| | | | | Thermo- | Amount |
|---|---|---|---|---|---|
| Abbrev. | Enzyme | Organism | Accession # | stable | (mg/mL) |
| GsRbsK | ribokinase | Geobacillus | WP_100664378.1 | Yes | 0.05 |
| | | stearothermophilus | | | |
| TkPrs | ribose-phosphate | Thermococcus | WP_011251185.1 | Yes | 0.25 |
| | diphosphokinase | kodakarensis | | | |
| EcPncB | nicotinate | Escherichia coli | WP_213200612.1 | No | 0.15 |
| | phosphoribosyl- | | | | |
| | transferase | | | | |
| GsNadD | nicotinate-nucleotide | Geobacillus | WP_053532206.1 | Yes | 0.05 |
| | adenylyltransferase | stearothermophilus | | | |
| EcNadE | NH3-dependent | Escherichia coli | WP_061355304.1 | No | 0.10 |
| | NAD⁺ synthetase | | | | |

### Cell-Free Isobutanol Production by an EM22 Reaction Mixture

The EM22 reaction mixture uses the six enzymes GsRbsK, TkPrs, EcPncB, GsNadD, EcNadE and EcNadK to biosynthesize NADP⁺ from ribose, nicotinate, NH₄⁺ and ATP. The composition of the EM22 mixture is summarized in Table 13 (below).

TABLE 13
EM22 mixture

| | | |
|---|---|---|
| TmHex | 0.07 | mg/ml |
| TmPgi | 0.2 | mg/ml |
| EcPfkB | 0.2 | mg/ml |
| GsFba | 0.35 | mg/ml |
| TmTpi | 0.07 | mg/ml |
| TkGapN | 0.46 | mg/ml |
| GsGapDHM6 | 0.1 | mg/ml |
| TmPgk | 0.1 | mg/ml |
| TtdgPgm | 0.18 | mg/ml |
| GsEno | 0.3 | mg/ml |
| TtPyk | 0.15 | mg/ml |
| BsAlsS | 0.15 | mg/ml |
| GsllvC | 1.7 | mg/ml |
| SmllvD | 0.9 | mg/ml |
| KivD | 0.07 | mg/ml |
| EcYahK | 0.27 | mg/ml |
| GsRbsK | 0.05 | mg/ml |
| TkPrs | 0.25 | mg/ml |
| EcPncB | 0.15 | mg/ml |
| GsNadD | 0.05 | mg/ml |
| EcNadE | 0.1 | mg/ml |
| EcNadK | 0.1 | mg/ml |
| Bis-tris propane pH 8.5 | 100 | mM |
| KCl | 10 | mM |
| MgCl₂ | 12.5 | mM |
| Glutathione | 1 | mM |
| Glucose-1-phosphate | 0.75 | mM |
| TPP | 0.5 | mM |
| PO₄ | 5 | mM |
| ATP | 10 | mM |
| Ribose | 3 | mM |
| Nicotinate | 3 | mM |
| NH₄⁺ | 3 | mM |

| | |
|---|---|
| Poly-P | 0 to 10 μl in 200 ul reaction |
| Cellulosic hydrolysate | 30% |

The reactions are set at 32 C for 16 h. 6 μL of added poly-P was used for ATP regeneration in NADP⁺ biosynthesis.

### Results

FIG. 6B depicts an exemplary plot of absorption at 340 nm over time showing that production of 6PGDL in the presence of the substrates ribose, nicotinate, ATP, and NH⁴⁺, and the five enzymes listed in Table 10, proceeds according to the biosynthetic reactions of FIG. 6A.

As shown by the results summarized in Table 14, added nicotinate, ribose, and ammonia can be converted in situ to NAD, and the resulting NAD can be converted by EcNadK in situ to produce NADP⁺ which can function as the redox carrier to maintain isobutanol production in a cell-free system.

TABLE 14

| | Enzyme Mixture | | | | | | |
|---|---|---|---|---|---|---|---|
| | EM18 | EM22 | EM22 | EM22 | EM22 | EM22 | EM22 |
| Sugar | C6 | C6 | C6 | C6 | C6 | C6 | C6 |
| NAD+ | 3 | 0 | 0 | 0 | 0 | 0 | 0 |
| (mM) | | | | | | | |
| NAD+ | 0 | 3 mM | 3 mM | 3 mM | 3 mM | 3 mM | 3 mM |
| Substrates | | each: | each: | each: | each: | each: | each: |
| | | ribose, | ribose, | ribose, | ribose, | ribose, | ribose, |
| | | nicotinate, | nicotinate, | nicotinate, | nicotinate, | nicotinate, | nicotinate, |
| | | NH₄⁺ | NH₄⁺ | NH₄⁺ | NH₄⁺ | NH₄⁺ | NH₄⁺ |
| EcNadK | 0.1 | 0.1 | 0.1 | 0.1 | 0.1 | 0.1 | 0.1 |
| (mg/mL) | | | | | | | |
| Poly-P (μL) | 0 | 0 | 2 | 4 | 6 | 8 | 10 |
| IB (g/L) | 20.2 | 16.6 | 19.8 | 20.5 | 21.7 | 20.7 | 20.9 |

These results demonstrate that the combination of six enzymes GsRbsK, TkPrs, EcPncB, GsNadD, EcNadE and EcNadK, and the substrates, ribose, nicotinate, ATP, and NH⁴⁺, can function as the sole redox carrier in a cell-free system to maintain isobutanol production without added NADP.

Notwithstanding the appended claims, the disclosure set forth herein is also defined by the following numbered embodiments, which may be beneficial alone or in combination, with one or more of the other embodiments or features disclosed herein. Without limiting the foregoing description, each of the individually numbered embodiments may be used or combined with any of the preceding or following numbered embodiments. Thus, this is intended to provide support for all such combinations and is not necessarily limited to specific combinations explicitly provided below:

Embodiment 1: A cell-free process for NADP-dependent biosynthesis of a compound, the process comprising contacting in a cell-free solution under suitable reaction conditions: an NADP-dependent enzyme, a substrate for the NADP-dependent enzyme, ATP, and (a) NAD, and a NAD kinase; (b) NMN, a NMN adenyltransferase, and a NAD kinase; and/or (c) ribose, nicotinate, NH⁴⁺, and a NAD kinase; whereby, NADP is produced in the solution and used by the NADP-dependent enzyme in the conversion of the substrate to the compound or to a precursor of the compound.

Embodiment 2: The process of Embodiment 1, wherein the cell-free solution does not comprise NADP or NADPH prior to contacting with (a) NAD, and a NAD kinase; (b) NMN, a NMN adenyltransferase, and a NAD kinase; and/or (c) ribose, nicotinate, NH⁴⁺, and a NAD kinase.

Embodiment 3: The process of any one of Embodiments 1-2, wherein the NAD kinase is selected from: EcNadK (WP_001059169.1), GsppnK1 (WP_011232266.1), GsppnK2 (WP_011230331.1), BliNadK (WP_009329373.1), TkNadK (WP_011251074.1), TmNadK (WP_004082250.1), PhNadK (WP_010885160.1), AfNadK (WP_010879860.1), CsNadK (WP_009610251.1), AtNadK (WP_003518725.1), ToNadK (WP_012572594.1), UTR1 (NP_012583.1), MtNadK (WP_003408383.1), BsNadK (WP_106610845.1), and a functional variant having at least 90%, at least 91%, at least 92%, at least 93%, at least 94%, at least 95%, at least 96%, at least 97%, at least 98%, or at least 99% amino acid sequence identity to the amino acid sequence of any of the preceding NAD kinases.

Embodiment 4: The process of any one of Embodiments 1-3, wherein the NMN adenyltransferase is selected from: ScNma1 (NP_013432.1), ScNma2 (NP_011524.1), POF1 (NP_009883.1), RnNmnat2 (NP_001041507.1), BtNmnat1 (NP_001069302.1), BtNmnat2 (XP_005217345.1), MmNmnat1 (NP_001343286.1), MmNmnat2 (NP_780669.1), MmNmnat3 (NP_653116.1), HsNmnat1 (NP_001284707.1), HsNmnat2 (NP_055854.1), HsNmnat3 (NP_001307440.1), AtNmnat (NP_200392.3), and a functional variant having at least 90%, at least 91%, at least 92%, at least 93%, at least 94%, at least 95%, at least 96%, at least 97%, at least 98%, or at least 99% amino acid sequence identity to the amino acid sequence of any of the preceding NMN adenyltransferases.

Embodiment 5: The process of any one of Embodiments 1-3, wherein the cell-free solution further comprises the enzymes: RbsK, Prs, PncB, NadD, and NadE; optionally, wherein the enzymes are GsRbsK (ALA70174.1), TkPrs (WP_011251185.1), EcPncB (WP_001307697.1), GsNadD (WP_053532206.1), and EcNadE (EFI2440128.1).

Embodiment 6: The process of any one of Embodiments 1-5, wherein the cell-free solution further comprises: (a) a precursor substrate, and one or more enzymes capable of converting the precursor substrate to the substrate for the NADP-dependent enzyme; and/or (b) one or more enzymes capable of converting the precursor of the compound produced by the NADP-dependent enzyme to the compound.

Embodiment 7: The process of any one of Embodiments 1-6, wherein the cell-free solution further comprises a precursor substrate selected from a C6 sugar, a C5 sugar, and a mixture thereof.

Embodiment 8: The process of Embodiment 7, wherein the precursor substrate is selected from glucose, ribose, xylose, and a combination thereof.

Embodiment 9: The process of any one of Embodiments 1-8, wherein the compound is selected from isobutanol, 3-methyl-1-butanol, leucine, and valine.

Embodiment 10: The process of Embodiment 9, wherein the compound in isobutanol.

Embodiment 11: The process of any one of Embodiments 1-10, wherein the cell-free solution further comprises an enzyme selected from: Hexokinase (Hex); Phosphoglucoisomerase (Pgi); Phosphoglucokinase (PfkB); Fructose 1,6 biphosphate aldolase (FBA); Triose phosphate isomerase (TPI); Non-phosphorylating glyceraldehyde 3 phosphate dehydrogenase (GapN); Phosphorylating glyceraldehyde 3 phosphate dehydrogenase (GapDHM6); Phosphoglycerate kinase (PGK); Phosphoglycerate mutase (PGM); Phosphoenolpyruvate hydratase (Eno); Pyruvate kinase (Pyk); Acetolactate synthase (AlsS); Ketol-acid reductoisomerase (IIvC); Dihydroxy-acid dehydratase (IIvD); Keto-isovalerate decarboxylase (KivD); Alcohol dehydrogenase (YahK); Ribokinase (RbsK); Ribose-phosphate diphosphokinase (Prs); Nicotinate phosphoribosyltransferase (PncB); Nicotinate-nucleotide adenylyltransferase (NadD); NH3-dependent NAD+ synthetase (NadE); Nicotinamide phosphoribosyltransferase (NAMPT); Polyphosphate kinase (Ppk); and any combination thereof.

Embodiment 12: The process of Embodiment 11, wherein the enzyme is selected from: TmHex (AAD36537.1), TmPgi (WP_004081585.1), EcPfkB (EFJ64522.1), GsFBA (KOR93738.1), TmTPI (AAA67520.1), TkGapN (WP_011249656.1), GsGapDHM6 (KOR95271.1), TmPGK (AAD35771.1), TtdgPGM (WP_011026502.1), GsEno (KOR95275), TtPyk (WP_011227631.1), BsAlsS (WP_003244057.1), GslIvC (WP_033014337.1), SmllvD (ESS16062.1), KivD (WP_085624856.1), EcYahK (WP_128424329.1), GsRbsK (ALA70174.1), TkPrs (WP_011251185.1), EcPncB (WP_001307697.1), GsNadD (WP_053532206.1), EcNadE (EFI2440128.1), NAMPT (NP_067499.2), Ppk (ABM08865.1), and a combination thereof.

Embodiment 13: A composition for NADP-dependent biosynthesis comprising a cell-free solution, wherein the cell-free solution comprises: (a) a NAD kinase, NAD, and ATP; (b) a NMN adenyltransferase, a NAD kinase, NMN and ATP; and/or (c) a NAD kinase, ribose, nicotinate, NH⁴⁺ and ATP.

Embodiment 14: The composition of Embodiment 13, wherein the cell-free solution does not comprise NADP or NADPH.

Embodiment 15: The composition of any one of Embodiments 13-14, wherein the NAD kinase is selected from: EcNadK (WP_001059169.1), GsppnK1 (WP_011232266.1), GsppnK2 (WP_011230331.1), BliNadK (WP_009329373.1), TkNadK (WP_011251074.1), TmNadK (WP_004082250.1), PhNadK (WP_010885160.1), AfNadK (WP_010879860.1), CsNadK (WP_009610251.1), AtNadK (WP_003518725.1), ToNadK (WP_012572594.1), UTR1 (NP_012583.1), MtNadK (WP_003408383.1), BsNadK (WP_106610845.1), and a functional variant having at least 90%, at least 91%, at least 92%, at least 93%, at least 94%, at least 95%, at least 96%, at least 97%, at least 98%, or at least 99% amino acid sequence identity to the amino acid sequence of any of the preceding NAD kinases.

Embodiment 16: The composition of any one of Embodiments 13-15, wherein the NMN adenyltransferase is selected from: ScNma1 (NP_013432.1), ScNma2 (NP_011524.1), POF1 (NP_009883.1), RnNmnat2 (NP_001041507.1), BtNmnat1 (NP_001069302.1), BtNmnat2 (XP_005217345.1), MmNmnat1 (NP_001343286.1), MmNmnat2 (NP_780669.1), MmNmnat3 (NP_653116.1), HsNmnat1 (NP_001284707.1), HsNmnat2 (NP_055854.1), HsNmnat3 (NP_001307440.1), AtNmnat (NP_200392.3), and a functional variant having at least 90%, at least 91%, at least 92%, at least 93%, at least 94%, at least 95%, at least 96%, at least 97%, at least 98%, or at least 99% amino acid sequence identity to the amino acid sequence of any of the preceding NMN adenyltransferases.

Embodiment 17: The composition of any one of Embodiments 13-16, wherein the cell-free solution further comprises the enzymes: RbsK, Prs, PncB, NadD, and NadE; optionally, wherein the enzymes are GsRbsK (ALA70174.1), TkPrs (WP_011251185.1), EcPncB (WP_001307697.1), GsNadD (WP_053532206.1), and EcNadE (EFI2440128.1).

Embodiment 18: The composition of any one of Embodiments 13-17, wherein the cell-free solution further comprises: an NADP-dependent enzyme; a substrate for the NADP-dependent enzyme; and ATP.

Embodiment 19: The composition of any one of Embodiments 13-18, wherein the cell-free solution further comprises: (a) a precursor substrate, and one or more enzymes capable of converting the precursor substrate to the substrate for the NADP-dependent enzyme; and/or (b) one or more enzymes capable of converting the precursor of the compound produced by the NADP-dependent enzyme to the compound.

Embodiment 20: The composition of any one of Embodiments 13-19, wherein the cell-free solution further comprises an enzyme selected from: Hexokinase (Hex); Phosphoglucoisomerase (Pgi); Phosphoglucokinase (PfkB); Fructose 1,6 biphosphate aldolase (FBA); Triose phosphate isomerase (TPI); Non-phosphorylating glyceraldehyde 3 phosphate dehydrogenase (GapN); Phosphorylating glyceraldehyde 3 phosphate dehydrogenase (GapDHM6); Phosphoglycerate kinase (PGK); Phosphoglycerate mutase (PGM); Phosphoenolpyruvate hydratase (Eno); Pyruvate kinase (Pyk); Acetolactate synthase (AlsS); Ketol-acid reductoisomerase (IIvC); Dihydroxy-acid dehydratase (IIvD); Keto-isovalerate decarboxylase (KivD); Alcohol dehydrogenase (YahK); Ribokinase (RbsK); Ribose-phosphate diphosphokinase (Prs); Nicotinate phosphoribosyltransferase (PncB); Nicotinate-nucleotide adenylyltransferase (NadD); NH3-dependent NAD+ synthetase (NadE); Nicotinamide phosphoribosyltransferase (NAMPT); Polyphosphate kinase (Ppk); and any combination thereof.

Embodiment 21: The composition of Embodiment 20, wherein the enzyme is selected from: TmHex (AAD36537.1), TmPgi (WP_004081585.1), EcPfkB (EFJ64522.1), GsFBA (KOR93738.1), TmTPI (AAA67520.1), TkGapN (WP_011249656.1), GsGapDHM6 (KOR95271.1), TmPGK (AAD35771.1), TtdgPGM (WP_011026502.1), GsEno (KOR95275), TtPyk (WP_011227631.1), BsAlsS (WP_003244057.1), GslIvC (WP_033014337.1), SmllvD (ESS16062.1), KivD (WP_085624856.1), EcYahK (WP_128424329.1), GsRbsK (ALA70174.1), TkPrs (WP_011251185.1), EcPncB (WP_001307697.1), GsNadD (WP_053532206.1), EcNadE (EFI2440128.1), NAMPT (NP_067499.2), Ppk (ABM08865.1), and a combination thereof.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. A cell-free process for NADP-dependent biosynthesis of a compound, the process comprising contacting in a cell-free solution under suitable reaction conditions: an NADP-dependent enzyme, a substrate for the NADP-dependent enzyme, ATP, and
(a) NAD, and a NAD kinase;
(b) NMN, a NMN adenyltransferase, and a NAD kinase; and/or
(c) ribose, nicotinate, NH⁴⁺, and a NAD kinase;
whereby, NADP is produced in the solution and used by the NADP-dependent enzyme in the conversion of the substrate to the compound or to a precursor of the compound.

2. The process of claim 1, wherein the cell-free solution does not comprise NADP or NADPH prior to contacting with (a) NAD, and a NAD kinase; (b) NMN, a NMN adenyltransferase, and a NAD kinase; and/or (c) ribose, nicotinate, NH⁴⁺, and a NAD kinase.

3. The process of claim 1, wherein the NAD kinase is selected from: EcNadK (WP_001059169.1), GsppnK1 (WP_011232266.1), GsppnK2 (WP_011230331.1), BliNadK (WP_009329373.1), TkNadK (WP_011251074.1), TmNadK (WP_004082250.1), PhNadK (WP_010885160.1), AfNadK (WP_010879860.1), CsNadK (WP_009610251.1), AtNadK (WP_003518725.1), ToNadK (WP_012572594.1), UTR1 (NP_012583.1), MtNadK (WP_003408383.1), BsNadK (WP_106610845.1), and a mixture thereof.

4. The process of claim 1, wherein the NMN adenyltransferase is selected from: ScNma1 (NP_013432.1), ScNma2 (NP_011524.1), POF1 (NP_009883.1), RnNmnat2 (NP_001041507.1), BtNmnat1 (NP_001069302.1), BtNmnat2 (XP_005217345.1), MmNmnat1 (NP_001343286.1), MmNmnat2 (NP_780669.1), MmNmnat3 (NP_653116.1), HsNmnat1 (NP_001284707.1), HsNmnat2 (NP_055854.1), HsNmnat3 (NP_001307440.1), AtNmnat (NP_200392.3), and a mixture thereof.

5. The process of claim 1, wherein the cell-free solution further comprises the enzymes: RbsK, Prs, PncB, NadD, and NadE; optionally, wherein the enzymes are GsRbsK (ALA70174.1), TkPrs (WP_011251185.1), EcPncB (WP_001307697.1), GsNadD (WP_053532206.1), and EcNadE (EFI2440128.1).

6. The process of claim 1, wherein the cell-free solution further comprises:
(a) a precursor substrate, and one or more enzymes capable of converting the precursor substrate to the substrate for the NADP-dependent enzyme;
(b) one or more enzymes capable of converting the precursor of the compound produced by the NADP-dependent enzyme to the compound;
(c) a precursor substrate selected from a C6 sugar, a C5 sugar, and a mixture thereof; optionally, wherein the precursor substrate is selected from glucose, ribose, xylose, and a combination thereof; and/or
(d) an enzyme selected from: Hexokinase (Hex); Phosphoglucoisomerase (Pgi); Phosphoglucokinase (PfkB); Fructose 1,6 biphosphate aldolase (FBA); Triose phosphate isomerase (TPI); Non-phosphorylating glyceraldehyde 3 phosphate dehydrogenase (GapN); Phosphorylating glyceraldehyde 3 phosphate dehydrogenase (GapDHM6); Phosphoglycerate kinase (PGK); Phosphoglycerate mutase (PGM); Phosphoenolpyruvate hydratase (Eno); Pyruvate kinase (Pyk); Acetolactate synthase (AlsS); Ketol-acid reductoisomerase (IIvC); Dihydroxy-acid dehydratase (IIvD); Keto-isovalerate decarboxylase (KivD); Alcohol dehydrogenase (YahK); Ribokinase (RbsK); Ribose-phosphate diphosphokinase (Prs); Nicotinate phosphoribosyltransferase (PncB); Nicotinate-nucleotide adenylyltransferase (NadD); NH3-dependent NAD+ synthetase (NadE); Nicotinamide phosphoribosyltransferase (NAMPT); Polyphosphate kinase (Ppk); and any combination thereof.

7. The process of claim 1, wherein the compound is selected from isobutanol, 3-methyl-1-butanol, leucine, and valine.

8. A composition for NADP-dependent biosynthesis comprising a cell-free solution, wherein the cell-free solution comprises:
(a) a NAD kinase, NAD, and ATP;
(b) a NMN adenyltransferase, a NAD kinase, NMN and ATP; and/or
(c) a NAD kinase, ribose, nicotinate, NH⁴⁺ and ATP.

9. The composition of claim 8, wherein the cell-free solution does not comprise NADP or NADPH.

10. The composition of claim 8, wherein the NAD kinase is selected from: EcNadK (WP_001059169.1), GsppnK1 (WP_011232266.1), GsppnK2 (WP_011230331.1), BliNadK (WP_009329373.1), TkNadK (WP_011251074.1), TmNadK (WP_004082250.1), PhNadK (WP_010885160.1), AfNadK (WP_010879860.1), CsNadK (WP_009610251.1), AtNadK (WVP_003518725.1), ToNadK (WP_012572594.1), UTR1 (NP_012583.1), MtNadK (WP_003408383.1), and BsNadK (WP_106610845.1).

11. The composition of claim 8, wherein the NMN adenyltransferase is selected from: ScNma1 (NP_013432.1), ScNma2 (NP_011524.1), POF1 (NP_009883.1), RnNmnat2 (NP_001041507.1), BtNmnat1 (NP_001069302.1), BtNmnat2 (XP_005217345.1), MmNmnat1 (NP_001343286.1), MmNmnat2 (NP_780669.1), MmNmnat3 (NP_653116.1), HsNmnat1 (NP_001284707.1), HsNmnat2 (NP_055854.1), HsNmnat3 (NP_001307440.1), and AtNmnat (NP_200392.3).

12. The composition of claim 8, wherein the cell-free solution further comprises the enzymes: RbsK, Prs, PncB, NadD, and NadE; optionally, wherein the enzymes are GsRbsK (ALA70174.1), TkPrs (WP_011251185.1), EcPncB (WP_001307697.1), GsNadD (WP_053532206.1), and EcNadE (EFI2440128.1).

13. The composition of claim 8, wherein the cell-free solution further comprises: an NADP-dependent enzyme; a substrate for the NADP-dependent enzyme; and ATP.

14. The composition of claim 8, wherein the cell-free solution further comprises:
(a) a precursor substrate, and one or more enzymes capable of converting the precursor substrate to the substrate for the NADP-dependent enzyme;
(b) one or more enzymes capable of converting the precursor of the compound produced by the NADP-dependent enzyme to the compound;
(c) a precursor substrate selected from a C6 sugar, a C5 sugar, and a mixture thereof; optionally, wherein the precursor substrate is selected from glucose, ribose, xylose, and a combination thereof; and/or
(d) an enzyme selected from: Hexokinase (Hex); Phosphoglucoisomerase (Pgi); Phosphoglucokinase (PfkB); Fructose 1,6 biphosphate aldolase (FBA); Triose phosphate isomerase (TPI); Non-phosphorylating glyceraldehyde 3 phosphate dehydrogenase (GapN); Phosphorylating glyceraldehyde 3 phosphate dehydrogenase (GapDHM6); Phosphoglycerate kinase (PGK); Phosphoglycerate mutase (PGM); Phosphoenolpyruvate hydratase (Eno); Pyruvate kinase (Pyk); Acetolactate synthase (AlsS); Ketol-acid reductoisomerase (IIvC); Dihydroxy-acid dehydratase (IIvD); Keto-isovalerate decarboxylase (KivD); Alcohol dehydrogenase (YahK); Ribokinase (RbsK); Ribose-phosphate diphosphokinase (Prs); Nicotinate phosphoribosyltransferase (PncB); Nicotinate-nucleotide adenylyltransferase (NadD); NH3-dependent NAD+ synthetase (NadE); Nicotinamide phosphoribosyltransferase (NAMPT); Polyphosphate kinase (Ppk); and any combination thereof.


SEQUENCE LISTING (50 sequences)

SEQ ID NO: 1 - protein, 292 aa, Escherichia coli
MNNHFKCIGIVGHPRHPTALTTHEMLYRWLCTKGYEVIVEQQIAHELQLKNVKTGTLAEIGQLADLAVVVGGDGNMLGAARTLARYDIKVIGINRGNLGFLTDLDPDNAQQQLADVLEGHYISEKRFLLEAQVCQQDCQKRISTAINEVVLHPGKVAHMIEFEVYIDEIFAFSQRSDGLIISTPTGSTAYSLSAGGPILTPSLDAITLVPMFPHTLSARPLVINSSSTIRLRFSHRRNDLEISCDSQIALPIQEGEDVLIRRCDYHLNLIHPKDYSYFNTLSTKLGWSKKLF

SEQ ID NO: 2 - protein, 267 aa, Geobacillus stearothermophilus
MDMERNRLYFFYKRDDKLIERVKPLIELAERGPFVVVDDYREANIIVSIGDDGAFLQAVRQTGFLPDRLYVGVSVLPARGFYCDFHIDDIDHMVEAAKNWKLEVRRYPIIEVTIDGAASFFCLNECSIRSQIIKTMAIDVFIDDLHFETFRGDGIIVSTPTGSTGYNKSVHGAVVDPLLPCFQVSELASLNSNRYRTLGSPFILSGSRTLTLKMSEETSHFPIIGLDNEALSIQHIERIDIRLSDRVVKTVRLKDNSFWDKVKRVFL

SEQ ID NO: 3 - protein, 271 aa, Geobacillus stearothermophilus
MKRTDAPLVFAITSKGDDISNALAQKMKTYLLDFDLRYDEEEPDLVISVGGDGTLLYAFHRYCHRLDKTAFVGVHTGHLGFYADWVPEELEKLVIAIAKTPYQVVEYPLLEVTIRYLNGGSEAKYLALNECTVKCVSGTLVMDVEIRGDLFERFRGDGLCISTPTGSTAYNKALGGAILHPSLEAIQVTEMASINNRVFRTIGSPLVLPAHHTCLLKPVNHVDFQITIDHLSLLHKEVKSIQCRVADEKVRFARFRPFPFWRRVRDSFIAD

SEQ ID NO: 4 - protein, 265 aa, Bacillus licheniformis
MTDRRNIYFFYKKSEETDEQCKKLKQLAEEHGFRVVHHHNEANIIASIGGDGTFLQAVRKTNFRDDCLYVGVAKKGKAHLYADFNIYDTDKMIEATNSEQIEVRKYPLIDVTVDGTSFQCLNEVSIRSSIIKTFVMDVYIDDLHFETFRGDGMIVSTPTGSTAYNKSVDGAIVDPLIPCIQVTELASLNNNTYRTLGSPFILSADRKLTLKIVQDGNDYPIIGLDNEAFSTMNVKEVEVSLSGKMIKTIKLKDNSFWEKVRRTFL

SEQ ID NO: 5 - protein, 278 aa, Thermococcus kodakarensis
MKFGVVARRDKLEALKLAYRVYDFLKVSGFDVVVDEDTYRYLGEFSEDDVLPLEEFDVDIIVVIGGDGTILRVEHKTKKEIPILGINMGTLGFLTEVEPHETFFALSRVIEGDYHIDERIKLRTFLDGENRVPDALNEVAVLTGIPGKIIHLKYYIDGGLADEVRADGLIISTPTGSTGYAMSAGGPFVDPRLDVVVIAPLAPIALSSRPMVVPSSSRIDVRNVAMTREVILSVDGQFYTYLSPETEITIVRSPRKTKFVRFNREIYPKYTMKIKSRF

SEQ ID NO: 6 - protein, 258 aa, Thermotoga maritima
MKIAILYREEREKEGEFLKEKISKEHEVIEFGEANAPGRVTADLIVVVGGDGTVLKAAKKAADGTPMVGFKAGRLGFLTSYTLDEIDRFLEDLRNWNFREETRWFIQIESELGNHLALNDVTLERDLSGKMVEIEVEVEHHSSMWFFADGVVISTPTGSTAYSLSIGGPIIFPECEVLEISPIAPQFFLTRSVVIPSNFKVVVESQRDINMLVDGVLTGKTKRIEVKKSRRYVRILRPPEYDYVTVIRDKLGYGRRIE

SEQ ID NO: 7 - protein, 277 aa, Pyrococcus horikoshii
MKFGIVARRDKEEALKLAYRVYDFLKVHGYEVVVDKETYEHFPHFKEGDVIPLDEFDVDFIVAIGGDGTILRIEHMTKKDIPILSINMGTLGFLTEVEPSDTFFALNRLIEGEYYIDERIKVRTYIDGENRVPDALNEVAILTGIPGKIIHMKYYVDGGLADEVRADGLVVSTPTGSTGYAMSAGGPFIDPRLDVILIAPLLPLPKTSVPMVIPGSSRIDIRMLTDREIILAIDGQYYEHLPPNVEITVVKSPRKTKFIRFTREIYPKYTIRIKERH

SEQ ID NO: 8 - protein, 249 aa, Archaeoglobus fulgidus
MRAAVVYKTDGHVKRIEEALKRLEVEVELFNQPSEELENFDFIVSVGGDGTILRILQKLKRCPPIFGINTGRVGLLTHASPENFEVELKKAVEKFEVERFPRVSCSAMPDVLALNEIAVLSRKPAKMIDVALRVDGVEVDRIRCDGFIVATQIGSTGYAFSAGGPVVEPYLECFILIPIAPFRFGWKPYVVSMERKIEVIAEKAIVVADGQKSVDFDGEITIEKSEFPAVFFKNEKRFRNLFGKVRSIG

SEQ ID NO: 9 - protein, 283 aa, Caldanaerobacter subterraneus
MKKVGVIPNINKDKDLEVTKSVVNWLLDHGSEPYLNEIVAARIGYEKHGKKANEIYSKSDFLIALGGDGTILNVARLCAPFGTPILAVNLGHLGFLTEIDASELFPSLEKIYKGEYAIEKRMMLEANVVKNDMEVINFRALNDIVITRGAFSRMARIKAYVNDNYVDTYLADGVIVATPTGSTAYSLSAGGPIVYPTVEVIIITPICPHTLYSRSIVVSPDDVIRLEIAEENQDLMITTDGQQGYKIDYRDVIYIKKSNEYTNLIKVKNSNFFDLLRDKLTER

SEQ ID NO: 10 - protein, 289 aa, Clostridium thermocellum
MLKIGIIPNEDKDEELKYTRILVDSIKKCGGTAIVCDDIALKLGDKESNINEDNIVDMSDVMVCLGGDGTFLKAARMTVVKGKPLLGVNLGKLGFLADVDKNDIENAVKRLVEDKFTVDERMMLDTVIVRDGKIIAEDIVLNDVVISRGAISRILHLKTYINDAFMDLYPGDGLIISTPTGSTAYSLSAGGPLVEPDVDLIICTPICPHLLYSRSFITTADRVIKVVVAESSSHEAMVTVDGQNGYEVRGGDVIITKKSRIRMPMVRLNGKNFFDVLRGKIYDRGESMK

SEQ ID NO: 11 - protein, 278 aa, Thermococcus onnurineus
MKFGIVARRDKEAALKLAYRVYDFLKVSGYEVYVDTDTYKYLPEFHEEDVLPLEDFDVDFIIVIGGDGTILRVEHKTKKEIPLLGINMGTLGFLTEVEPHEAFFALSKLIEGDYHIDERIKLRTYLNGENVVPDALNEVAILTGIPGKIIHLRYYIDEGLADEIRADGLIVSTPTGSTGYAMSAGGPFVDPRLDVIVIAPLAPIALSSRPMIVPSYTKIDVRNLAVTREIILAIDGQFYTYLEPETEITIRLSPRKTKFVRFTNEVYPKYTMKIKRKF

SEQ ID NO: 12 - protein, 530 aa, Saccharomyces cerevisiae
MKENDMNNGVDKWVNEEDGRNDHHNNNNNLMKKAMMNNEQIDRTQDIDNAKEMLRKISSESSSRRSSLLNKDSSLVNGNANSGGGTSINGTRGSSKSSNTHFQYASTAYGVRMLSKDISNTKVELDVENLMIVTKLNDVSLYFLTRELVEWVLVHFPRVTVYVDSELKNSKKFAAGELCEDSKCRESRIKYWTKDFIREHDVFFDLVVTLGGDGTVLFVSSIFQRHVPPVMSFSLGSLGFLTNFKFEHFREDLPRIMNHKIKTNLRLRLECTIYRRHRPEVDPNTGKKICVVEKLSTHHILNEVTIDRGPSPFLSMLELYGDGSLMTVAQADGLIAATPTGSTAYSLSAGGSLVCPTVNAIALTPICPHALSFRPIILPESINLKVKVSMKSRAPAWAAFDGKDRIELQKGDFITICASPYAFPTVEASPDEFINSISRQLNWNVREQQKSFTHILSQKNQEKYAHEANKVRNQAEPLEVIRDKYSLEADATKENNNGSDDESDDESVNCEACKLKPSSVPKPSQARFSV

SEQ ID NO: 13 - protein, 307 aa, Mycobacterium tuberculosis
MTAHRSVLLVVHTGRDEATETARRVEKVLGDNKIALRVLSAEAVDRGSLHLAPDDMRAMGVEIEVVDADQHAADGCELVLVLGGDGTFLRAAELARNASIPVLGVNLGRIGFLAEAEAEAIDAVLEHVVAQDYRVEDRLTLDVVVRQGGRIVNRGWALNEVSLEKGPRLGVLGVVVEIDGRPVSAFGCDGVLVSTPTGSTAYAFSAGGPVLWPDLEAILVVPNNAHALFGRPMVTSPEATIAIEIEADGHDALVFCDGRREMLIPAGSRLEVTRCVTSVKWARLDSAPFTDRLVRKFRLPVTGWRGK

SEQ ID NO: 14 - protein, 266 aa, Bacillus subtilis
MKFAVSSKGDQVSDTLKSRIQAYLLDFDMELDENEPEIVISVGGDGTLLYAFHRYSDRLDKTAFVGVHTGHLGFYADWVPHEIEKLVLAIAKTPYHTVEYPLLEVIVTYHENEREERYLALNECTIKSIEGSLVADVEIKGQLFETFRGDGLCLSTPSGSTAYNKALGGAIIHPSIRAIQLAEMASINNRVFRTVGSPLLLPSHHDCMIKPRNEVDFQVTIDHLTLLHKDVKSIRCQVASEKVRFARFRPFPFWKRVQDSFIGKGE

SEQ ID NO: 15 - protein, 258 aa, Saccharomyces cerevisiae
MKKTFEQFRKSNLLFQVLKGPQHLECQKLFVLDSSFNPPHLAHFQLLSQTIKNFKLKDTRSHVLLLLAVNNADKLPKPASFPTRLEMMCLFADYLQEKLPQSVVSVGLTVFSKFIDKDKILHEQFVKGCSADIGYLVGFDTIARIFDEKYYHPLKISDVMESFMSGSQLYCLARGDCHLSAESQLRYASDILEGKFEPVIPREWGARIHVMQNDYPALRNVSSSEIRNKLKNGQVESLKDELPLCIYDYLINNKTIFD

SEQ ID NO: 16 - protein, 401 aa, Saccharomyces cerevisiae
MDPTRAPDFKPPSADEELIPPPDPESKIPKSIPIIPYVLADANSSIDAPFNIKRKKKHPKHHHHHHHSRKEGNDKKHQHIPLNQDDFQPLSAEVSSEDDDADFRSKERYGSDSTTESETRGVQKYQIADLEEVPHGIVRQARTLEDYEFPSHRLSKKLLDPNKLPLVIVACGSFSPITYLHLRMFEMALDAISEQTRFEVIGGYYSPVSDNYQKQGLAPSYHRVRMCELACERTSSWLMVDAWESLQPSYTRTAKVLDHFNHEINIKRGGVATVTGEKIGVKIMLLAGGDLIESMGEPNVWADADLHHILGNYGCLIVERTGSDVRSFLLSHDIMYEHRRNILIIKQLIYNDISSTKVRLFIRRAMSVQYLLPNSVIRYIQEHRLYVDQTEPVKQVLGNKE

SEQ ID NO: 17 - protein, 395 aa, Saccharomyces cerevisiae
MDPTKAPDFKPPQPNEELQPPPDPTHTIPKSGPIVPYVLADYNSSIDAPFNLDIYKTLSSRKKNANSSNRMDHIPLNTSDFQPLSRDVSSEEESEGQSNGIDATLQDVTMTGNLGVLKSQIADLEEVPHTIVRQARTIEDYEFPVHRLTKKLQDPEKLPLIIVACGSFSPITYLHLRMFEMALDDINEQTRFEVVGGYFSPVSDNYQKRGLAPAYHRVRMCELACERTSSWLMVDAWESLQSSYTRTAKVLDHFNHEINIKRGGIMTVDGEKMGVKIMLLAGGDLIESMGEPHVWADSDLHHILGNYGCLIVERTGSDVRSFLLSHDIMYEHRRNILIIKQLIYNDISSTKVRLFIRRGMSVQYLLPNSVIRYIQEYNLYINQSEPVKQVLDSKE

SEQ ID NO: 18 - protein, 307 aa, Rattus norvegicus
MTETTKTHVILLACGSFNPITKGHIQMFERARDYLHKTGRFIVIGGIVSPVHDSYGKQGLVSSRHRLIMCQLAVQNSDWIRVDPWECYQDTWQTTCSVLEHHRDLMKRVTGCILSNVNTPSMTPVIGQPQHENTQPIYQNSNVPTKPTAAKILGKVGESLGRICCVRPPVERFTFVDENANLGTVMRYEEIELRILLLCGSDLLESFCIPGLWNEADMEVIVGDFGIVVVPRDAADADRIMNHSSILRKYKNNIMVVKDDINHPMSVVSSTKSRLALQHGDGHVVDYLSQPVIDYILKSQLYINASG

SEQ ID NO: 19 - protein, 281 aa, Bos taurus
MENSEKTEVVLLACGSFNPITNMHLRLFELAKDYMNGTGKYKVIKGIISPVGDAYKKKGLISAYHRVIMAELATKNSKWVEVDTWESLQKEWTETAKVLRHHQEKLEASICDPQQNSPVLEKPGRKRKWAEQKQDISEKKSLEQTKTKGVPKVKLLCGADFLESFGVPNLWKSEDITKILGDYGLICITRAGNDAQKFIYESDVLWKHQNNIHLVNEWITNDISSTKIRRALRRGQSIRYLVPDLVEEYIEKHNLYSSESEERNVGVVLAPLQRNTTEVKA

SEQ ID NO: 20 - protein, 348 aa, Bos taurus
MYRSPSLQYHTSDVELSDSIKHIETQKEKRPLGQREGPARAMTETTKTHVILLACGSFNPITKGHIQMFERARDYLHKTGRFIVIGGIVSPVHDSYGKQGLVSSRHRLIMCQLAVQNSDWIRVDPWECYQDTWQTTCSVLEHHRDLMKRVTGCILSNVNTPSMTPVIGQPRNETSQPIYQNNNAPSKPTAAKILGKVGESLSRICCVRPPVERFTFVDENANLGTVMRYEEIELRILLLCGSDLLESFCIPGLWNEADMEVIVGDFGIVVVPRDAADTDRIMNHSSILRKYKNNIMVVKDDINHPMSVVSSTKSRLALQHGDGHVVDYLSQPVIDYILKSQLYINASG

SEQ ID NO: 21 - protein, 285 aa, Mus musculus
MDSSKKTEVVLLACGSFNPITNMHLRLFELAKDYMHATGKYSVIKGIISPVGDAYKKKGLIPAHHRIIMAELATKNSHWVEVDTWESLQKEWVETVKVLRYHQEKLATGSCSYPQSSPALEKPGRKRKWADQKQDSSPQKPQEPKPTGVPKVKLLCGADLLESFSVPNLWKMEDITQIVANFGLICITRAGSDAQKFIYESDVLWRHQSNIHLVNEWITNDISSTKIRRALRRGQSIRYLVPDLVQEYIEKHELYNTESEGRNAGVTLAPLQRNAAEAKHNHSTL

SEQ ID NO: 22 - protein, 307 aa, Mus musculus
MTETTKTHVILLACGSFNPITKGHIQMFERARDYLHKTGRFIVIGGIVSPVHDSYGKQGLVSSRHRLIMCQLAVQNSDWIRVDPWECYQDTWQTTCSVLEHHRDLMKRVTGCILSNVNTPSMTPVIGQPQHENTQPIYQNSNVPTKPTAAKILGKVGESLSRICCVRPPVERFTFVDENANLGTVMRYEEIELRILLLCGSDLLESFCIPGLWNEADMEVIVGDFGIVVVPRDAADTDRIMNHSSILRKYKNNIMVVKDDINHPMSVVSSTKSRLALQHGDGHVVDYLSQPVIDYILKSQLYINASG

SEQ ID NO: 23 - protein, 245 aa, Mus musculus
MKNRIPVVLLACGSFNPITNMHLRLFEVARDHLHQTGRYQVIEGIISPVNDSYGKKDLVASHHRVAMARLALQTSDWIRVDPWESEQAQWMETVKVLRHHHRELLRSSAQMDGPDPSKTPSASAALPELKLLCGADVLKTFQTPNLWKDTHIQEIVEKFGLVCVSRSGHDPERYISDSPILQQFQHNIHLAREPVLNEISATYVRKALGQGQSVKYLLPEAVITYIRDQGLYINDGSWKGKGKTG

SEQ ID NO: 24 - protein, 279 aa, Homo sapiens
MENSEKTEVVLLACGSFNPITNMHLRLFELAKDYMNGTGRYTVVKGIISPVGDAYKKKGLIPAYHRVIMAELATKNSKWVEVDTWESLQKEWKETLKVLRHHQEKLEASDCDHQQNSPTLERPGRKRKWTETQDSSQKKSLEPKTKAVPKVKLLCGADLLESFAVPNLWKSEDITQIVANYGLICVTRAGNDAQKFIYESDVLWKHRSNIHVVNEWIANDISSTKIRRALRRGQSIRYLVPDLVQEYIEKHNLYSSESEDRNAGVILAPLQRNTAEAKT

SEQ ID NO: 25 - protein, 307 aa, Homo sapiens
MTETTKTHVILLACGSFNPITKGHIQMFERARDYLHKTGRFIVIGGIVSPVHDSYGKQGLVSSRHRLIMCQLAVQNSDWIRVDPWECYQDTWQTTCSVLEHHRDLMKRVTGCILSNVNTPSMTPVIGQPQNETPQPIYQNSNVATKPTAAKILGKVGESLSRICCVRPPVERFTFVDENANLGTVMRYEEIELRILLLCGSDLLESFCIPGLWNEADMEVIVGDFGIVVVPRDAADTDRIMNHSSILRKYKNNIMVVKDDINHPMSVVSSTKSRLALQHGDGHVVDYLSQPVIDYILKSQLYINASG

SEQ ID NO: 26 - protein, 252 aa, Homo sapiens
MKSRIPVVLLACGSFNPITNMHLRMFEVARDHLHQTGMYQVIQGIISPVNDTYGKKDLAASHHRVAMARLALQTSDWIRVDPWESEQAQWMETVKVLRHHHSKLLRSPPQMEGPDHGKALFSTPAAVPELKLLCGADVLKTFQTPNLWKDAHIQEIVEKFGLVCVGRVGHDPKGYIAESPILRMHQHNIHLAKEPVQNEISATYIRRALGQGQSVKYLIPDAVITYIKDHGLYTKGSTWKGKSTQSTEGKTS

SEQ ID NO: 27 - protein, 238 aa, Arabidopsis thaliana
MDVPLPVEKLSYGSNTEDKTCVVLVATGSFNPPTFMHLRMFELARDELRSKGFHVLGGYMSPVNDAYKKKGLLSAEHRLEMCNVSCQSSDFVMVDPWEASQSNYQRTLTVLSRVKTFLTTNRHVPEESLKVMLLCGSDLLLSFCTPGVWIPEQLRTICKDYGIVCIRREGQDVENMISGDEILNENCANVKIVDNTVPNQISSSRLRQCISRGLSVKYLTEDGVIDYIRQHQLYTELT

SEQ ID NO: 28 - protein, 317 aa, Thermotoga maritima
MPKLKLIGVDLGGTTFSVGLVSEDGKILKKVTRDTLVENGKEDVIRRIAETILEVSDGEEAPYVGIGSPGSIDRENGIVRFSPNFPDWHNVPLTDELAKRTGKKVFLENDANAFVLGEKWFGAGRGHDHIVALTLGTGIGGGVVTHGYLLTGRDGIGAELGHVVVEPNGPMCNCGTRGCLEAVASATAIRRFLREGYKKYHSSLVYKLAGSPEKADAKHLFDAARQGDRFALMIRDRVVDALARAVAGYIHIFNPEIVIIGGGISRAGEILFGPLREKVVDYIMPSFVGTYEVVASPLVEDAGILGAASIIKERIGG

SEQ ID NO: 29 - protein, 448 aa, Thermotoga maritima
MSLKFDFSNLFEPNISGGLTDEDVKSVEEKVTSAVRNFVENTPDFAKLDRSWIDSVKSLEDWIINFDTVVVLGIGGSGLGNLALHYSLRPLNWNEMTREERNGYARVFVVDNVDPDLMSSVLDRIDPKTTLFNVISKSGSTAEVMATYSIARGILEAYGLDPREHMLITTDPEKGFLRKLVKEEGFRSLEVPPGVGGRFSVLTPVGLLSAMAEGIDIDELHEGAKDAFEKSMKENILENPAAMIALTHYLYLNKGKSISVMMAYSNRMIYLVDWYRQLWAESLGKRYNLKGEEVFTGQTPVKALGATDQHSQIQLYNEGPNDKVITFLRVENFDREIVIPETGRAELSYLARKKLSELLLAEQTGTEEALRENNRPNMRVTFDGLTPYNVGQFFAYYEAATAFMGYLLEINPFDQPGVELGKKITFALMGREGYTYEIKERSKKVIIE

SEQ ID NO: 30 - protein, 310 aa, Escherichia coli
MMVRIYTLTLAPSLDSATITPQIYPEGKLRCTAPVFEPGGGGINVARAIAHLGGSATAIFPAGGATGEHLVSLLADENVPVATVEAKDWTRQNLHVHVEASGEQYRFVMPGAALNEDEFRQLEEQVLEIESGAILVISGSLPPGVKLEKLTQLISAAQKQGIRCIVDSSGEALSAALAIGNIELVKPNQKELSALVNRELTQPDDVRKAAQEIVNSGKAKRVVVSLGPQGALGVDSENCIQVVPPPVKSQSTVGAGDSMVGAMTLKLAENASLEEMVRFGVAAGSAATLNQGTRLCSHDDTQKIYAYLSR

SEQ ID NO: 31 - protein, 287 aa, Geobacillus stearothermophilus
MSLVSMKEMLNEALRGKYAVGQFNINNLEWTQAILAAAEEEKSPVILGVSEGAARYMGGFKTVVNMVKGLMEDMNITVPVAIHLDHGSSFEKCKAAIDAGFTSVMIDASHHPFEENVRITSQVVEYAHARGVSVEAELGIVGGQEDDVVGEGVIYADPKECEELVKRTGIDCLAPALGSVHGPYKGEPKLGFAEMEQIRDLTGIPLVLHGGTGIPTEQIQRAISLGTSKINVNTENQIAFTKAVRELLAKDPNVYDPRKIIGPGRDAIKATVIGKMREFGSSGKAAQ

SEQ ID NO: 32 - protein, 255 aa, Thermotoga maritima
ITRKLILAGNWKMHKTISEAKKFVSLLVNELHDVKEFEIVVCPPFTALSEVGEILSGRNIKLGAQNVFYEDQGAFTGEISPLMLQEIGVEYVIVGHSERRRIFKEDDEFINRKVKAVLEKGMTPILCVGETLEEREKGLTFCVVEKQVREGFYGLDKEEAKRVVIAYEPVWAIGTGRVATPQQAQEVHAFIRKLLSEMYDEETAGSIRILYGGSIKPDNFLGLIVQKDIDGGLVGGASLKESFIELARIMRGVIS

SEQ ID NO: 33 - protein, 507 aa, Thermococcus kodakarensis
MVEPFVPEGEIFEGIFRQNEGIPEFATYVNGEWVFTGKTAEVRSPIDGSLIARVSLSDMALSNRAVAAAYSAGRHEIRDTPGEKRLEAFLKVAELIRDSFDDFVTALVLDAGKPLSNARGEVTATIERLEKTTMEFGRLIGDYIPGDWSAESLGSEGIVKREPYGVVLAISPYNYPLFISTAKIVPALLAGNAVLLKPSSQDPLAPLLLSRVLQLAGIPESAYHLLTVPGALMDSILADRRIRAVTFTGSTEVGEHILSMGGIKFYHMELGGKDPAVVLDDAPLEETVEKLVKGMVSYSGQRCDAIRLIIAEEGIYEQLKRELVAALSKIEPENPLEDEDAIMGPLINERSAEKIEEVYRDALEKGAVPLTGFKRKGAYVWPVLLEASREVLPGLRAFQEDVFGPLTILVKVSNEDEAVELANSSRFGLDAAVFSGDDSRARKVARRLEVGAVFINEFPRHGIGYYPFGGMKDSGIGREGIGYSIETLTTTKTIVRNYRGRGVWDYI

SEQ ID NO: 34 - protein, 335 aa, Geobacillus stearothermophilus
MAVKVGINGFGRIGRNVFRAALKNPDIEVVAVNDLTDANTLAHLLKYDSVHGRLDAEVSVNGNNLVVNGKEIIVKAERDPENLAWGEIGVDIVVESTGRFTKREDAAKHLEAGAKKVIISAPAKNEDITIVMGVNQDKYDPKAHHVISNASCTTNCLAPFAKVLHEQFGIVRGMMTTVHSYTNDQRILDLPHKDLRRARAAAESIIPTTTGAAKAVALVLPELKGKLNGMAMRVPTPNVSVVDLVAELEKEVTVEEVNAALKAAAEGELKGILAYSEEPLVSRDYNGSTVSSTIDALSTMVIDGKMVKVVSWYDNETGYSHRVVDLAAYIASKGL

SEQ ID NO: 35 - protein, 654 aa, Thermotoga maritima
MEKMTIRDVDLKGKRVIMRVDFNVPVKDGVVQDDTRIRAALPTIKYALEQGAKVILLSHLGRPKGEPSPEFSLAPVAKRLSELLGKEVKFVPAVVGDEVKKAVEELKEGEVLLLENTRFHPGETKNDPELAKFWASLADIHVNDAFGTAHRAHASNVGIAQFIPSVAGFLMEKEIKFLSKVTYNPEKPYVVVLGGAKVSDKIGVITNLMEKADRILIGGAMMFTFLKALGKEVGSSRVEEDKIDLAKELLEKAKEKGVEIVLPVDAVIAQKIEPGVEKKVVRIDDGIPEGWMGLDIGPETIELFKQKLSDAKTVVWNGPMGVFEIDDFAEGTKQVALAIAALTEKGAITVVGGGDSAAAVNKFGLEDKFSHVSTGGGASLEFLEGKELPGIASIADKKKITRKLILAGNWKMHKTISEAKKFVSLLVNELHDVKEFEIVVCPPFTALSEVGEILSGRNIKLGAQNVFYEDQGAFTGEISPLMLQEIGVEYVIVGHSERRRIFKEDDEFINRKVKAVLEKGMTPILCVGETLEEREKGLTFCVVEKQVREGFYGLDKEEAKRVVIAYEPVWAIGTGRVATPQQAQEVHAFIRKLLSEMYDEETAGSIRILYGGSIKPDNFLGLIVQKDIDGGLVGGASLKESFIELARIMRGVIS

SEQ ID NO: 36 - protein, 249 aa, Thermus thermophilus
MYKVVLLRHGESIWNMENRFTGWTDVDLSPKGIEEAKQAGKILKEKGFTFDAAFTSVLKRAIRTLWIVLDELDLMWIPVYKSWRLNERHYGALQGLNKAETAKKYGEEQVKLWRRSAEVRPPALTKDDPRYPGNDPRYADLSEDEIPLTENLIDTINRVIPYWESTIAPTIKSGKRVIIAAHGNSLRGLVKYLDNLSNEEIMELNIPTGIPLVYELDENLKPIRHYYLADEEELKKKQQEVAEQGKARA

SEQ ID NO: 37 - protein, 430 aa, Geobacillus stearothermophilus
MSAIIDVYAREVLDSRGNPTVEVEVYTEEGGFGRALVPSGASTGEYEAVELRDGDKNRYLGKGVLKAVENVNEIIAPEIIGLEVTDQVAIDRALIELDGTENKSKLGANAILGVSLAVARAAADELGLPLYHYLGGFNAKTLPVPMMNILNGGAHADNNVDIQEFMIMPVGAESFREALRMGAEIFHSLKAVLKSKGYNTAVGDEGGFAPNLKSNEEALQTIIEAIEKAGYKPGEQVMLAMDVASSELYNKEDGKYHLEGEGVVKTSEEMVAWYEELVSKYPIISIEDGLDENDWEGHKLLTERLGKKVQLVGDDLFVTNTKKLAEGIEKGVGNSILIKVNQIGTLTETFDAIEMAKRAGYTAVVSHRSGETEDSTIADIAVATNAGQIKTGAPSRTDRVAKYNQLLRIEDELGHTAIYQGIRSFYNLKK

SEQ ID NO: 38 - protein, 474 aa, Thermus thermophilus
MPPFKRTKIVATLGPATDDKEVIRALAEAGADVFRLNFSHGAPEDHRRRVGWVREVAEELGRTLAVLQDLQGPKIRVGRFREGQVLLRPGQRFVLTAEPVEGDEHRVSVSYKGLPEDVSPGQILLLDDGRIRLKVLEVRSPEILTEVEVGGVLSNNKGINIPGADLSIPALSEKDIQDLALGAELGVDWVAVSFVRTRDDLLLARHYLSRYGSKARLMAKIEKPSAVARFEEILEEADGIMVARGDLGVEMPLEEVPIVQKRLILRCIAAGKPVITATQMLESMVQNPSPTRAEASDVANAIFDGTDAVMLSAETAAGAYPVEAVAMMARIAKAVESSPEFLQKLNVLRPAPTPTTQDAIAQAADDVVEAVGARAIVVFTATGGSARRIARTRPQVPILALTPNPEVRNQLALVWGVYPHLAPDPQDTDDMVRIALREVKALGLAQVGDRVVIAAGVPFGVRGTTNLIRVERVG

SEQ ID NO: 39 - protein, 570 aa, Bacillus subtilis
MTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 40 - protein, 341 aa, Geobacillus stearothermophilus
MAKVYYNGDANEQYLQGKTVAIIGYGSQGHAHAQNLRDSGVRVIVGLRKGKSWEQAEQDGFEVYSVREAAKQADIVMVLLPDEKQPAVYKEEIEPGLEPGNALVFAHGFNIHFSQIVPPEHVDVFLVAPKGPGHLVRRTYAEGAGVPALIAVYQDVTGHAKETALAYAKAIGAARAGVLETTFKEETETDLFGEQAVLCGGLTALIKAGFETLVEAGYQPEVAYFECLHEMKLIVDLLYEGGLSWMRYSISDTAQWGDFITGPRIINDAVKAEMKKVLDDIQTGKFAKSWILENQANRPEFNAINRRENEHLIEVVGRELRSMMPFVKAKQKEAVVPGAKH

SEQ ID NO: 41 - protein, 578 aa, Streptococcus mutans
MMKGSGQMTDKKTLKDLRNRSSVYDSMVKSPNRAMLRATGMQDEDFEKPIVGVISTWAENTPCNIHLHDFGKLAKVGVKEAGAWPVQFGTITVSDGIAMGTQGMRFSLTSRDIIADSIEAAMGGHNADAFVAIGGCDKNMPGSVIAMANMDIPAIFAYGGTIAPGNLDGKDIDLVSVFEGVGHWNHGDMTKEEVKALECNACPGPGGCGGMYTANTMATAIEVLGLSLPGSSSHPAESAEKKADIEEAGRAVVKMLEMGLKPSDILTREAFEDAITVTMALGGSTNSTLHLLAIAHAANVELTLDDFNTFQEKVPHLADLKPSGQYVFQDLYKVGGVPAVMKYLLKNGFLHGDRITCTGKTVAENLKAFDDLTPGQKVIMPLENPKREDGPLIILHGNLAPDGAVAKVSGVKVRRHVGPAKVFNSEEEAIEAVLNDDIVDGDVVVVRFVGPKGGPGMPEMLSLSSMIVGKGQGEKVALLTDGRFSGGTYGLVVGHIAPEAQDGGPIAYLQTGDIVTIDQDTKELHFDISDEELKHRQETIELPPLYSRGVLGKYAHIVSSASRGAVTDFWKPEETGKK

SEQ ID NO: 42 - protein, 548 aa, Lactococcus lactis
MYTVGDYLLDRLHELGIEEIFGVPGDYNLQFLDQIISRKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDETLPSFLGIYNGKLSEPNLKEFVESADFILMLGVKLTDSSTGAFTHHLNENKMISLNIDEGKIFNESIQNFDFESLIYSLLNLSEIEYKGKYIDKKQENFVPSNALLSQDRLWQAVENLTQSNETIVAEQGTSFFGASSIFLKPKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELVLAKEDAPKVLKKMGKLFAEQNKS

SEQ ID NO: 43 - protein, 349 aa, Escherichia coli
MKIKAVGAYSAKQPLEPMDITRREPGPNDVKIEIAYCGVCHSDLHQVRSEWAGTVYPCVPGHEIVGRVVAVGDQVEKYAPGDLVGVGCIVDSCKHCEECEDGLENYCDHMTGTYNSPTPDEPGHTLGGYSQQIVVHERYILRIRHPQEQLAAVAPLLCAGITTYSPLRHWQAGPGKKVGVVGIGGLGHMGIKLAHAMGAHVVAFTTSEAKREAAKALGADEVVNSRNADEMAAHLKSFDFILNTVAAPHNLDDFTTLLKRDGTMTLVGAPATPHKSPEVFNLIMKRRAIAGSMIGGIPETQEMLDFCAEHGIVADIEMIRADQINEAYERMLRGDVKYRFVIDNRTLTD

SEQ ID NO: 44 - protein, 297 aa, Geobacillus stearothermophilus
MNKPTITVIGSINMDLVTVAVRFPNQGETILGEQFLTTPGGKGANQAVAAARLGADVRMIGAVGDDAFGQELIRSLQNEGVSVDYVKPVTYEHTGIASITISERDNRIIVVPGANHALTPEDLDPYESVIAESDVCLLQLEIPLPVVERAVSIAYRHGVRVIVNPAPAQPLPLVVLEQASFLTPNEHERAILFDKMDEEAFADKLIVTEGAKGVRIWQDGQERLIPSFHVPVVDTTGAGDTFNGALAVALAEGKPLDEACRFANAAAALSVTKLGAQAGMPRRQEVESFLSKQKESH

SEQ ID NO: 45 - protein, 280 aa, Thermococcus kodakarensis
MFLLGSGGKHFEDELRNAGAKILEVEIKRFPDGEKYVRVMGNGDEATVVSSTFYPQDEKIVELLLLGDALREKGFEKLKLVVPYFAYSRQDRVTKDGEPISVRAVMRALGIYYEELYIFDTHNPETLRFFPGKAVNVSPARVIGEYFREKLGDGLVLAPDKGALERARAVAEVLGLEYSHFEKRRISPTEVEMHPVDVDVKGKNVLIVDDIISTGGTMVRAAELLRKLGAKKIYVSATHGVFAEGAIERVSRAVDELAVTNTIPTPVSRISIVPELLKLE

SEQ ID NO: 46 - protein, 400 aa, Escherichia coli
MTQFASPVLHSLLDTDAYKLHMQQAVFHHYYDVHVAAEFRCRGDDLLGIYADAIREQVQAMQHLRLQDDEYQWLSALPFFKADYLNWLREFRFNPEQVTVSNDNGKLDIRLSGPWREVILWEVPLLAVISEMVHRYRSPQADVAQALDTLESKLVDFSALTAGLDMSRFHLMDFGTRRRFSREVQETIVKRLQQESWFVGTSNYDLARRLSLTPMGTQAHEWFQAHQQISPDLANSQRAALAAWLEEYPDQLGIALTDCITMDAFLRDFGVEFASRYQGLRHDSGDPVEWGEKAIAHYEKLGIDPQSKTLVFSDNLDLRKAVELYRHFSSRVQLSFGIGTRLTCDIPQVKPLNIVIKLVECNGKPVAKLSDSPGKTICHDKAFVRALRKAFDLPHIKKAS

SEQ ID NO: 47 - protein, 216 aa, Geobacillus stearothermophilus
MGKIGILGGTFDPPHYGHLIMANEVLDALELSQIWFLPNRLPPHKQHEQVTKSEDRLRMLELAVAGHPYFRVETIELEREGPSYTYDTVQQLVAMHPDDEFYFIIGADMVEYLPNWHRIDELIKLVTFVGVKRPGYSMETPYPVVEVEVPQFAVSSSLIRQRVQNGQTIRYLVPEGVRLYIEEKGLYGARTGVADCETTVDGAALRAYAWRCRNGR

SEQ ID NO: 48 - protein, 276 aa, Escherichia coli
MTLQQQIIKALGAKPQINAEEEIRRSVDFLKSYLQTYPFIKSLVLGISGGQDSTLAGKLCQMAINELRLETGNESLQFIAVRLPYGVQADEQDCQDAIAFIQPDRVLTVNIKGAVLASEQALREAGIELSDFVRGNEKARERMKAQYSIAGMTSGVVVGTDHAAEAITGFFTKYGDGGTDINPLYRLNKRQGKQLLAALACPEHLYKKAPTADLEDDRPSLPDEVALGVTYDNIDDYLEGKNVPQQVARTIENWYLKTEHKRRPPITVFDDFWKKL

SEQ ID NO: 49 - protein, 491 aa, Mus musculus
MNAAAEAEFNILLATDSYKVTHYKQYPPNTSKVYSYFECREKKTENSKVRKVKYEETVFYGLQYILNKYLKGKVVTKEKIQEAKEVYREHFQDDVFNERGWNYILEKYDGHLPIEVKAVPEGSVIPRGNVLFTVENTDPECYWLTNWIETILVQSWYPITVATNSREQKKILAKYLLETSGNLDGLEYKLHDFGYRGVSSQETAGIGASAHLVNFKGTDTVAGIALIKKYYGTKDPVPGYSVPAAEHSTITAWGKDHEKDAFEHIVTQFSSVPVSVVSDSYDIYNACEKIWGEDLRHLIVSRSTEAPLIIRPDSGNPLDTVLKVLDILGKKFPVTENSKGYKLLPPYLRVIQGDGVDINTLQEIVEGMKQKKWSIENVSFGSGGALLQKLTRDLLNCSFKCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 50 - protein, 286 aa, Arthrobacter aurescens
MPMVAAVEFAKSPAEVLRVGSGFSLAGVDPESTPGYTGVKADGKALLAAQDARLAELQEKLFAEGKFGNPKRLLLILQAMDTAGKGGIVSHVVGAMDPQGVQLTAFKAPTDEEKSHDFLWRIEKQVPAAGMVGVFDRSQYEDVLIHRVHGWADAAELERRYAAINDFESRLTEQGTTIVKVMLNISKDEQKKRLIARLDDPSKHWKYSRGDLAERAYWDDYMDAYSVAFEKTSTEIAPWHVVPANKKWYARIAVQQLLLDALGGLQLDWPKADFDVAAERALVVES